(19) United States
(12) Patent Application Publication
(10) Pub. No.: US 2011/0165577 A1
(43) Pub. Date: Jul. 7, 2011

(54) SELECTION OF COLORECTAL CANCER PATIENTS FOR NEO-ADJUVANT AND ADJUVENT SYSTEMIC ANTI-CANCER TREATMENT

(76) Inventors: Nils Aage Brünner, Hellerup (DK); Hans Jørgen Nielsen, Kgs. Lyngby (DK)

(21) Appl. No.: 12/997,369

(22) PCT Filed: Jun. 11, 2009
(86) PCT No.: PCT/DK09/50124

§ 371 Date: Mar. 4, 2011

(30) Foreign Application Priority Data
Jun. 11, 2008 (DK) PA 2008 00809

(51) Int. Cl. C12Q 1/68; C12Q 1/37

(52) U.S. Cl. 435/6.12; 435/23

(57) ABSTRACT

The present invention relates to method for determining whether a gastrointestinal cancer patient is likely to experience a disease recurrence, said method comprising steps of determining concentration of TIMP-1 in a pre-operative plasma sample of said patient, and determining the concentration of CEA in a pre-operative body fluid sample of said patient, and evaluate whether the patient as likely to experience a disease recurrence. The present invention further provides kits for application of said methods.

### TECHNICAL FIELD OF THE INVENTION

The present invention relates to the field of cancer therapy. In particular, the present invention relates to methods to increase the selection of colorectal cancer patients for systemic anti-cancer treatment. In particular, the present invention relates to improvements in selection of lymph node negative colorectal cancer patients for systemic adjuvant therapy and selection of lymph node positive colorectal cancer patients for less aggressive adjuvant systemic therapy. In addition, the present invention relates to a method in which the efficacy of neo-adjuvant and adjuvant treatment of colorectal cancer patients can be biochemically evaluated.

### BACKGROUND OF THE INVENTION

Colorectal cancer (CRC), a form of gastrointestinal cancer, is the third most frequently appearing cancer and the second most frequent cause of cancer related mortality in the Western World. There are approximately 400,000 new cases annually.

Patients with stage III (lymph node positive) colorectal cancer are offered adjuvant systemic chemotherapy. Adjuvant treatment with either FULFOX or FULFIRI chemotherapeutic regimes results in an average improvement in survival of 30%. In contrast, the majority of patients with stage II (lymph node negative) colorectal cancer do not receive systemic adjuvant treatment. Knowing that approximately 15% of the stage II patients will experience disease recurrence, which is most often fatal, these patients are obvious candidates for adjuvant chemotherapy. However, without validated prognostic markers to subdivide the stage II patients into prognostic groups, 85% of the patients would be treated with adjuvant chemotherapy although they are cured by the primary surgical procedures. The recent ASCO recommendation for adjuvant treatment of stage II colorectal cancer concludes that at present there is no evidence for a beneficial effect of systemic adjuvant treatment of this patient group as a whole. However, due to lack of valid prognostic biomarkers in this patient group, there has been no attempt to investigate the beneficial effect of adjuvant chemotherapy in a “high risk” stage II patient group.

The ASCO 2006 update of recommendations for the use of tumor markers in gastrointestinal cancers does for the first time recommend preoperative serum carcinoembryonic antigen (CEA) to be analysed (Locker GY et al., JCO, 2006, 24: 5313-5327). It is stated that CEA may be ordered preoperatively in patients with colorectal carcinoma if it would assist in staging and surgical treatment planning. Although elevated preoperative CEA (>5 mg/mL) may correlate with poorer prognosis, data are insufficient to support the use of CEA to determine whether to treat a patient with adjuvant therapy.

Holten-Andersen et al. (2000) published that measurements of Tissue Inhibitor of Metalloproteinases 1 (TIMP-1) in preoperatively obtained plasma samples from 588 colorectal cancer patients gave highly statistically significant prognostic information. Of outmost importance was the fact that the prognostic information obtained by plasma TIMP-1 measurements was independent of stage of disease. Thus, the stage I and II patients (Dukes A and B patients) (n=217) could be divided into highly statistically significant prognostic groups. The 70 percentile of TIMP-1 values was used as the cut-off point which resulted in a Hazard Ratio (HR) of 2.1; 95% Confidence Interval (CI): 1.3-3.5 between low and high plasma TIMP-1 patients. In the same way, plasma TIMP-1 measurements could be used to stratify the Dukes stage C (stage III) patients (n=94) into statistically significant different prognostic groups (Holten-Andersen et al., Clin Cancer Res 2000).

Holten-Andersen et al. (2004) also performed a retrospective cohort study of 352 rectal cancer (RC) patients undergoing elective surgery, wherein plasma TIMP-1 was measured in samples obtained preoperatively. The study confirmed an independent association between plasma TIMP-1 levels and survival of node negative CRC patients.

In 2006, Holten-Andersen et al. report that by measuring plasma TIMP-1 in a sample obtained 6 months postoperatively (n=280), prognostic information can be obtained. The age and gender adjusted 95th percentile derived from a set of 808 blood donor plasmas was used as cut-off point to divide patients into low versus high plasma TIMP-1. Again, the prognostic impact of plasma TIMP-1 was independent of disease stage, meaning that patients in each individual disease stage could be stratified regarding survival by their postoperative plasma TIMP-1 level. Of particular interest was that the strong association between 6 month postoperative plasma TIMP-1 and survival also translated into an association between postoperative plasma TIMP-1 and time to local recurrence and time to distant metastases, respectively, supporting the prognostic impact of plasma TIMP-1.

In the same 2006 study, Holten-Andersen et al. also measured 6 months postoperative serum CEA levels. A weak but significant association between postoperative levels of TIMP-1 and CEA was found (p<0.0001; Spearman rank correlation=0.3). Multivariate analyses were performed with end-point of death of all causes, for postoperative TIMP-1 and CEA, including other recorded clinicopathological parameters. Scored as high or low according to specified cut points, TIMP-1 and CEA were found to be independent predictors of colorectal cancer patient survival. (TIMP-1: HR=1.6; 95% CI: 1.1-2.3; P=0.02; CEA: HR=3.7; 95% CI: 2.5-5.6; P<0.0001). Similar results were obtained for time to local recurrence and time to distant metastases. These results indicate that the prognostic information obtained by measurements of postoperative plasma TIMP-1 levels and postoperative serum CEA levels is additive, meaning that by measuring both TIMP-1 and CEA postoperatively, superior prognostic information can be obtained as compared with measurements of only one of these proteins. Accordingly, by measuring the postoperative plasma TIMP-1 levels and postoperative serum CEA levels the study indicates that valuable stage independent prognostic information can be obtained.

Pellegrini et al. 2000 disclose measurement of soluble CEA and TIMP-1 serum levels for use as markers of pre-invasive to invasive colorectal cancer. The study indicates that serum level of CEA 1 can be used as a progression marker for the transition from stage II to stage III and serum TIMP-1 levels can be used as a progression marker for the transition from stage III to stage IV As apparent from the statistical analysis serum levels of the parameters being studied (MMP-1, TIMP-1, CEA and p53 antibody) were analysed in relation to disease progression as a generic parameter, and in relation to specific stage transitions (I to II, II to III, III to IV) in order to highlight possible progression markers for these phases of disease. Thus, the authors do not study the different stages separately but merely the stage transitions. The authors describe that significant values were only observed for levels of sCEA (p=0.03) in the passage from stage II to stage III, and for levels of TIMP-1 (p=0.015) in the passage from stage III to stage 1V when correlation procedures were used. The authors find a statistically significant difference in CEA levels between patients with stage II-III disease and in TIMP-1 levels between stage III and IV patients.

Studying serum TIMP-1 levels in CRC patients, Ishida et al. (2003) did not find a close correlation with serum CEA levels, and the authors concluded from their study that measuring TIMP-1 would not help to predict the prognosis CRC patients.

At present, CEA is the only recommended serological biomarker to be used to guide treatment of colorectal cancer patients. CEA is approved for monitoring of colorectal cancer patients, allowing for early therapeutic intervention in the case of a rise in serum CEA levels in a patient with prior or current colorectal cancer.

### SUMMARY OF THE INVENTION

It has been discovered that analysis of both TIMP-1 and CEA and optionally at least one additional parameter in a preoperative patient sample provides independent prognostic indication of gastrointestinal cancer recurrence.

The inventors have previously presented two independent studies on plasma TIMP-1 as a stage independent prognostic marker in CRC patients (Holten-Andersen et al., Clin Cancer Res 2000 and Holten-Andersen et al., Eur J Cancer 2004). The inventors have disclosed two independent studies one hypothesis generating (Brünner et al., unpublished data (included in Example 1)) and one validation study (Brünner et al., unpublished data (included in Example 2)) on the additive effect by combining measurements of CEA and TIMP-1 in the prognostic separation of CRC patients.

The present invention pertains to mainly two aspects:

I. methods for determining whether a gastrointestinal cancer patient is likely to experience a disease recurrence or disease related death. Such methods are performed before adjuvant or neo-adjuvant anti-cancer treatment, and

II. methods for determining whether a gastrointestinal cancer patient is likely to experience a disease recurrence or disease related death following neo-adjuvant or adjuvant anti-cancer therapy.

Thus, the present invention allows for determining whether a gastrointestinal cancer patient is likely to experience a disease recurrence or disease related death before and after adjuvant or neo-adjuvant anti-cancer treatment.

I. Methods for determining whether a gastrointestinal cancer patient is likely to experience a disease recurrence or disease related death.

Accordingly, a first aspect of the present invention concern a method for determining whether a gastrointestinal cancer patient is likely to experience a disease recurrence or disease related death, said method comprising:
a) determining the total concentration of TIMP-1 in a pre-operative plasma sample of said patient,
b) determining the concentration of CEA in a pre-operative body fluid sample of said patient,
c) combining the concentration of TIMP-1 with the concentration of CEA to result in a combined TIMP-1/CEA parameter
d) indicating the patient as likely to experience a disease recurrence or disease related death if the combined TIMP-1/CEA parameter is at or above a predefined combined TIMP-1/CEA discriminating value, and
e) indicating the individual as unlikely to experience a disease recurrence or disease related death if the combined TIMP-1/CEA parameter is below said predefined TIMP-1/CEA combined discriminating value.

A second aspect concern a method for determining whether a gastrointestinal cancer patient is likely to experience a disease recurrence or disease related death, said methods comprising:
a) determining the total concentration of TIMP-1 in a pre-operative plasma sample of said patient,
b) determining the concentration of CEA in a pre-operative body fluid sample of said patient,
c) determining at least one additional parameter pre-operative of said patient,
d) combining the concentration of TIMP-1 with the concentration of CEA and the at least one additional parameter to result in a combined additional parameter,
e) indicating the patient as likely to experience a disease recurrence or disease related death if the combined additional parameter is at or above a predefined combined additional discriminating value, and
f) indicating the individual as unlikely to experience a disease recurrence or disease related death if the combined additional parameter is below said predefined combined additional discriminating value.

II. Methods for determining whether a gastrointestinal cancer patient is likely to experience a disease recurrence or disease related death following neo-adjuvant or adjuvant anti-cancer therapy.

A third aspect concerns a method for determining whether a gastrointestinal cancer patient is likely to experience a disease recurrence or disease related death following neo-adjuvant or adjuvant anti-cancer therapy, said method comprising
a) determining at a first time point the total concentration of TIMP-1 in a pre-operative plasma sample of said patient,
b) determining at said first time point the total concentration of CEA in a pre-operative plasma sample of said patient,
b) combining the concentration of TIMP-1 with the concentration of CEA to result in a combined TIMP-1/CEA parameter,
c) indicating the patient as likely to experience a disease recurrence or disease related death if the combined TIMP-1/CEA parameter is at or above a predefined combined TIMP-1/CEA discriminating value, and
d) indicating the individual as unlikely to experience a disease recurrence or disease related death if the combined TIMP-1/CEA parameter is below said predefined TIMP-1/CEA combined discriminating value,
e) if the individual is likely to experience a disease recurrence or disease related death determining at a second time point the total concentration of TIMP-1 in a post-operative plasma sample of said patient,
f) determining at said second time point the total concentration of CEA in a post-operative plasma sample of said patient,
g) combining the concentration of TIMP-1 with the concentration of CEA to result in a combined TIMP-1/CEA parameter,
h) indicating the patient as likely to experience a disease recurrence or disease related death if the combined TIMP-1/CEA parameter is at or above a predefined combined TIMP-1/CEA discriminating value, and
i) indicating the individual as unlikely to experience a disease recurrence or disease related death if the combined TIMP-1/CEA parameter is below said predefined TIMP-1/CEA combined discriminating value.

A fourth aspect concern methods for determining whether a gastrointestinal cancer patient is likely to experience a disease recurrence or disease related death following neo-adjuvant or adjuvant anti-cancer therapy, said methods comprising:
a) determining at a first time point the total concentration of TIMP-1 in a pre-operative plasma sample of said patient,
b) determining at said first time point the concentration of CEA in a pre-operative body fluid sample of said patient,
c) determining at said first time point at least one additional parameter pre-operatively of said patient,
d) combining the concentration of TIMP-1 with the concentration of CEA and the at least one additional parameter to result in a combined additional parameter,
e) indicating the patient as likely to experience a disease recurrence if the combined additional parameter is at or above a predefined combined additional discriminating value,
f) indicating the individual as unlikely to experience a disease recurrence or disease related death if the combined additional parameter is below said predefined combined additional discriminating value,
g) if the individual is likely to experience a disease recurrence or disease related death determining at a second time point the total concentration of TIMP-1 in a post-operative plasma sample of said patient,
h) determining at said second time point the concentration of CEA in a post-operative body fluid sample of said patient,
i) determining at said second time point at least one additional parameter post-operatively of said patient,
j) combining the concentration of TIMP-1 with the concentration of CEA and the at least one additional parameter to result in a combined additional parameter,
k) indicating the patient as likely to experience a disease recurrence if the combined additional parameter is at or above a predefined combined additional discriminating value, and
l) indicating the individual as unlikely to experience a disease recurrence or disease related death if the combined additional parameter is below said predefined combined additional discriminating value.

Accordingly, in one embodiment of the invention concerns the determination of changes in the concentrations of plasma levels of TIMP-1 and serum or plasma levels of CEA and optionally at least one additional parameter (e.g., the prognostic index solved by formula A) between a first time point and a second time point in the course of a patient's gastrointestinal cancer disease. By measuring the changes in the concentrations of plasma levels of TIMP-1 and serum or plasma levels of CEA and optionally at least one additional parameter (e.g., the prognostic index solved by formula A) between a first time point and a second time point in the course of a patient's gastrointestinal cancer disease one can determine whether a patient is likely to experience a disease recurrence or a disease related death. Such determination can be performed using any of the above disclosed methods—i.e. the methods for determining whether a gastrointestinal cancer patient is likely to experience a disease recurrence or disease related death following neo-adjuvant or adjuvant anti-cancer therapy.

The measurements of TIMP-1, CEA and optionally the at least one additional parameter can be made at various time points (including but not limited to the first and second time point) such as, for example, less than or equal to 1, 2, 3, 4, 5, 6, 7, 8, 9, 10, 11, 12, 13, 14, 15, 16, 17, 18, 19, 20, 21, 22, 23 or 24 months before or after a form of therapy (e.g., surgery, neo-adjuvant or adjuvant anti-cancer therapy), e.g. in the range of 1-24 months before or after a form of therapy (e.g., surgery, neo-adjuvant or adjuvant anti-cancer therapy) such as in the range of 2-23 months, e.g. in the range of 3-22 months, such as in the range of 4-21 months, e.g. in the range of 5-20 months, such as in the range of 6-19 months, e.g. in the range of 7-18 months, such as in the range of 8-17 months, e.g. in the range of 9-16 months, such as in the range of 10-15 months, e.g. in the range of 11-14 months, such as in the range of 12-13 months, e.g. in the range from 1-5 months, such as 2-6 months. Time points less than or equal to 1 month include but is not limited to 1 week, 2 weeks or 3 weeks. It is to be understood that these time points applies to the above disclosed methods (such as but not limited to the methods disclosed as the first, second and third and fourth aspect of the present invention). In another embodiment the therapy may include radiation, immunotherapy and/or chemotherapy.

Optionally, the aforementioned methods can include any one or more of the following steps: receiving a request to identify whether a patient sample from a gastrointestinal cancer patient contains markers that predict recurrence of gastrointestinal cancer; receiving a biological sample obtained from a preoperative gastrointestinal cancer patient; contacting a biological sample obtained from said selected patient with a first antibody to TIMP-1 or a probe that complements an mRNA encoding TIMP-1; contacting said biological sample obtained from said patient with a second antibody to CEA or a probe that complements an mRNA encoding CEA; determining a first value representing the amount of binding of said first antibody or the amount of TIMP-1 mRNA bound by the probe; determining a second value representing the amount of binding of said second antibody or the amount of CEA mRNA bound by the probe; inputting the first and second values into a computer configured to transform said first and second values into a prognostic index using an algorithm expressed by:

0.11×Log 2(CEA)+0.41×Log 2(TIMP-1)

determining whether said solved prognostic index is associated with recurrence of gastrointestinal cancer or remission of gastrointestinal cancer by comparing the solved prognostic index to a database containing a plurality of prognostic indices, wherein some of the indices are associated with a recurrence of gastrointestinal cancer and some of the indices are associated with remission of gastrointestinal cancer; and communicating said determination of whether said solved prognostic index is associated with recurrence of gastrointestinal cancer or remission of gastrointestinal cancer to said person making said request.

In the present application the algorithm expressed by “0.11×Log 2 (CEA)+0.41×Log 2 (TIMP-1)” may also be termed “Formula A”—thus the terms are used herein interchangeably.

A further aspect relates to a kit for determining whether a gastrointestinal cancer patient is likely to experience a disease recurrence or disease related death, said kit comprising
a) reagents for determining the total concentration of TIMP-1 in a pre-operative plasma sample of said patient, and
b) reagents for determining the concentration of CEA in a pre-operative body fluid sample of said patient, and optionally
c) reagents for determining the concentration of at least one additional tumour marker in a pre-operative body fluid sample of said patient.

Yet a further aspect relates to a kit for determining whether a gastrointestinal cancer patient is likely to experience a disease recurrence or disease related death following neo-adjuvant or adjuvant anti-cancer therapy, said methods comprising, said kit comprising
a) reagents for determining the total concentration of TIMP-1 in a pre-operative plasma sample of said patient, and
b) reagents for determining the concentration of CEA in a pre-operative body fluid sample of said patient, and optionally
c) reagents for determining the concentration of at least one additional tumour marker in a pre-operative body fluid sample of said patient.

### BRIEF DESCRIPTION OF THE FIGURES

FIG. 1.

Colorectal cancer (CRC) stage II and III populations.

FIG. 2. Hazard ratios based on the multivariable model (formula A) as a function of TIMP-1.

FIG. 3. Kaplan-Meier estimates of disease free survival for Dukes' B patients.

FIG. 4. Kaplan-Meier estimates of disease free survival for Dukes' C patients.

FIGS. 5 and 6. Flowcharts.

The present invention will now be described in more details in the following.

### DETAILED DESCRIPTION OF THE INVENTION

As described above, no prior publication has suggested that by combining preoperatively measured plasma TIMP-1 levels with serum or plasma CEA levels and optionally at least one additional parameter, superior prognostic information can be obtained for patients suffering from gastrointestinal cancer exemplified by colorectal cancer. That these two proteins could be combined into a common prognostic profile was therefore not obvious.

Several investigators have reported on the combination of serum CEA with other biomarkers e.g. CA242 and CA19-9 without being able to demonstrate an additive prognostic value of the two markers. The present inventors have also added other biomarkers than CEA to their TIMP-1 values without being able to demonstrate any additive effect.

Thus, it is not obvious that the specific combination of preoperative plasma TIMP-1 and preoperative serum CEA levels could be combined into a common prognostic profile (e.g. a TIMP-1/CEA parameter) being superior to measurements of each of the two biomarkers with regard to prognostic evaluation.

It may be argued that since the inventors previously have shown that 6 months postoperative plasma TIMP-1 and serum CEA levels could be combined it is obvious that the same could be done with the preoperatively obtained TIMP-1 and CEA values. However, as shown by Holten-Andersen et al., 2006, a significant number of the analysed patients change their plasma TIMP-1 values from the preoperative plasma sample to the postoperative plasma sample meaning that it was impossible to predict the value of combined TIMP-1 and CEA from the present literature. This is also true for CEA.

At present a large group of patients with stage II CRC do not receive adjuvant systemic anti-cancer therapy although it is known that approximately 15% of the patients will experience a disease recurrence or disease related death over 5 years postoperatively. In addition, approximately 30% of stage III CRC patients are cured by the initial surgical procedures (known from historical data), however, these patients are most often offered adjuvant systemic anti cancer therapy.

Evidently there is an unmet need for methods of identifying the prognostic group of the stage II and stage III CRC patients, who will benefit from adjuvant systemic anti cancer therapy.

It has been discovered by the present inventors that preoperative serum or plasma CEA levels combined with preoperative plasma TIMP-1 levels (e.g. the TIMP-1/CEA parameter) can be used to predict patient prognosis in stage II and stage III CRC patients. By analyzing both of these biomarkers, additive prognostic information that allows one to predict whether a patient will experience a recurrence of cancer can be obtained. Plasma TIMP-1 levels and serum or plasma CEA levels may be further combined with at least one additional parameter (e.g. the combined additional parameter).

Thus, unexpectedly, it was discovered that the amounts of CEA and TIMP-1 and optionally at least one additional parameter can be used to develop a prognostic index that can predict recurrence of CRC independent of the stage of disease. i.e. this additive prognostic information obtained by measuring both TIMP-1 and CEA is superior to the prognostic information that can be obtained by only measuring one of the proteins.

The following describes a large hypothesis generating study. The study surprisingly shows that the combination of TIMP-1 and CEA measurements in pre-operatively obtained blood yields significant additive prognostic information in patients suffering from stage I-III colorectal cancer. Thus, measurements of TIMP-1 and CEA provide superior prognostic information than each of TIMP-1 and CEA alone.

The following describes a large independent validation study including preoperatively sampled plasma from 319 patients. The patients were all treated in Sweden. The TIMP-1 assay established as described herein was used to determine the plasma TIMP-1 levels and a commercial CEA assay was used to determine CEA levels in corresponding sera from the patients. The biomarker analyses were performed in Copenhagen, Denmark. The TIMP-1 and CEA data were subsequently sent to Sweden and the clinical data to Denmark. At both locations, a correlation between plasma TIMP-1 and serum CEA and patient survival was carried out. Similar results were obtained: Plasma TIMP-1, as well as, serum CEA was stage independent prognostic markers when measured in preoperatively obtained blood samples from patients suffering from CRC. Accordingly, this study confirmed that the amounts of TIMP-1 and plasma CEA measured in preoperative plasma can be used to generate highly statistically significant information of patient prognosis in stage II, as well as, in stage III CRC patients (see also Example 2).

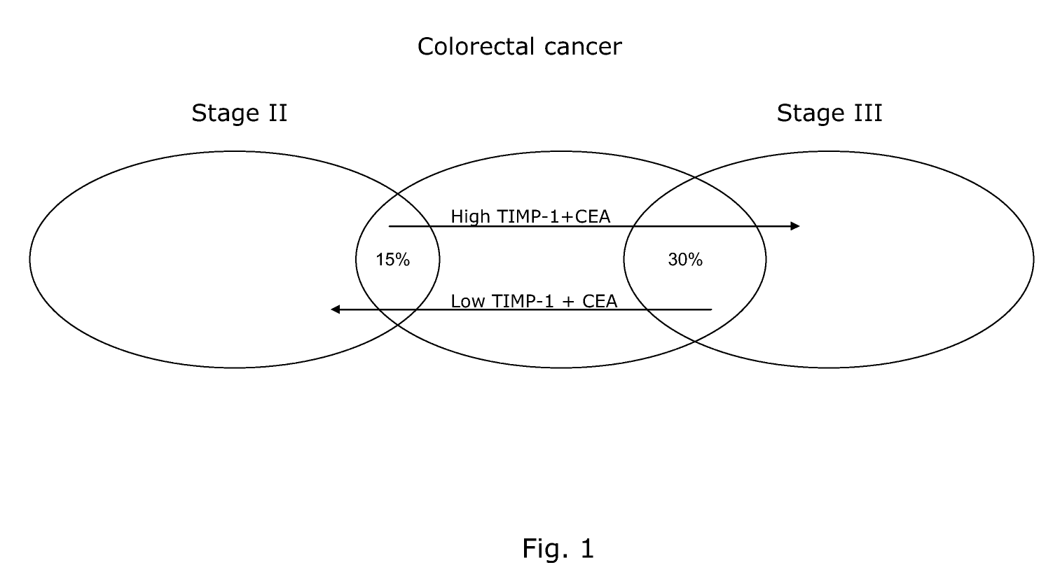

The present invention thus relates to improved methods to separate gastrointestinal cancer patients (exemplified by CRC patients) into prognostic groups, thereby guiding the use of systemic anti cancer therapy in stage I-III CRC patients (for stage II and III CRC patients see FIG. 1). Specifically, some embodiments relate to methods wherein at least one value representing the preoperative serum or plasma CEA level in a patient or a patient sample are combined with at least one value representing the preoperative serum or plasma TIMP-1 level in a patient or a patient sample. The two measured values are entered into a predefined algorithm (e.g., formula A) and the result represents a prognostic index or solved value that provides an accurate prognosis of recurrence of CRC. In addition, this algorithm allows for subsequent addition of independent prognostic variables such as tumor differentiation grade, genomic alterations (MSS/MSI, p53, ras, 18q-LOH/DCC), histological findings (grade and lymph and blood vessel invasion), demographic patient data such as gender, age and ethnicity and other biomarkers such as uncomplexed TIMP-1, TIMP-1 in complex with specific MMP's, soluble CD63, soluble uPA receptor (suPAR), YKL-40, CA19-9, p66 Shc, MMP's, ADAM's and kallekreins. It should be emphasised that the above markers may be measured in tumor tissue (either unfixed or fixed and/or in blood samples or other bodily fluids (urine, faeces, saliva, mucus, sweat). Furthermore, the algorithm may compensate for the fact that the level of TIMP-1 is age dependent, meaning that the level of TIMP-1 increases with increasing age. In addition, any relationships between biomarkers and demographic variables can be incorporated into such algorithm.

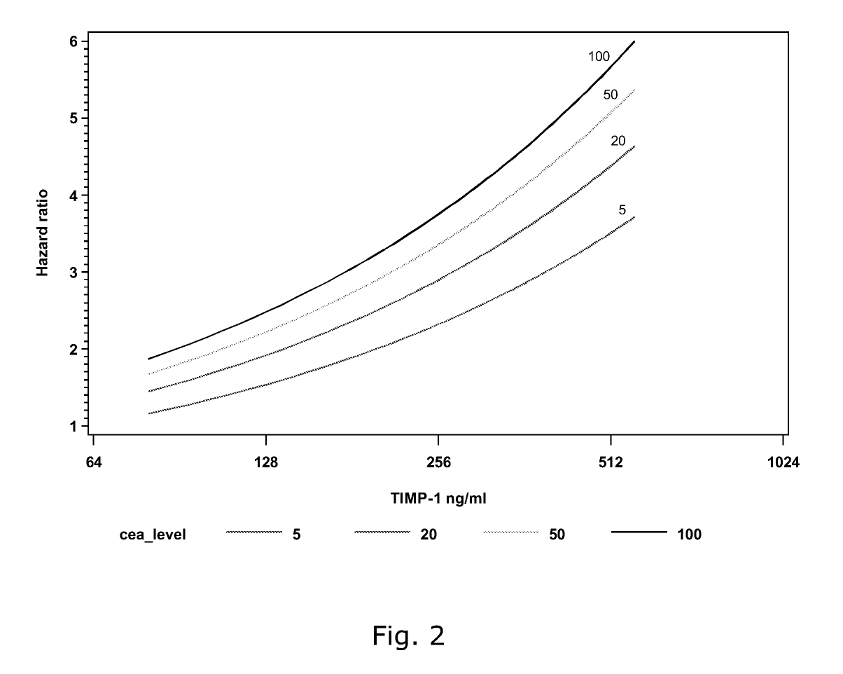

The algorithm (formula A): 0.11×Log 2 (CEA)+0.41×Log 2 (TIMP-1) can be extended by adding additional markers+YY×Log 2 (new marker R)+TT×log 2 (new marker Z) etc (for outcome see e.g. FIG. 2).

I. Methods for Determining Whether a Gastrointestinal Cancer Patient is Likely to Experience a Disease Recurrence or Disease Related Death.

A first aspect of the present invention relates to methods for determining whether a gastrointestinal cancer patient is likely to experience a disease recurrence or disease related death. This method comprises determining the total concentration of TIMP-1 in a pre-operative plasma sample and determining the concentration of CEA in a pre-operative body fluid sample both samples obtained from the patient. If the total concentration of TIMP-1 is at or above a predefined TIMP-1 discriminating value and at the same time the concentration of CEA is at or above a predefined CEA discriminating value it indicates that the patient is likely to experience a disease recurrence. If the total concentration of TIMP-1 is below said predefined TIMP-1 discriminating value and at the same time the concentration of CEA is below said predefined CEA discriminating value it indicates that the individual is unlikely to experience a disease recurrence.

Thus, in a preferred embodiment the present invention pertains to a method for determining whether a gastrointestinal cancer patient is likely to experience a disease recurrence or disease related death, said method comprising
a) determining the total concentration of TIMP-1 in a pre-operative plasma sample of said patient,
b) determining the concentration of CEA in a pre-operative body fluid sample of said patient,
c) combining the concentration of TIMP-1 with the concentration of CEA to result in a combined TIMP-1/CEA parameter,
d) indicating the patient as likely to experience a disease recurrence or disease related death if the combined TIMP-1/CEA parameter is at or above a predefined combined TIMP-1/CEA discriminating value, and
e) indicating the individual as unlikely to experience a disease recurrence or disease related death if the combined TIMP-1/CEA parameter is below said predefined TIMP-1/CEA combined discriminating value.

In another preferred embodiment the present invention pertains to a method for determining whether a gastrointestinal cancer patient is likely to experience a disease recurrence or disease related death, said method comprising
a) determining the total concentration of TIMP-1 in a pre-operative plasma sample of said patient,
b) determining the concentration of CEA in a pre-operative body fluid sample of said patient,
c) determining at least one additional parameter pre-operatively of said patient,
d) combining the concentration of TIMP-1 with the concentration of CEA and the at least one additional parameter to result in a combined additional parameter,
e) indicating the patient as likely to experience a disease recurrence or disease related death if the combined additional parameter is at or above a predefined combined additional discriminating value, and
f) indicating the individual as unlikely to experience a disease recurrence or disease related death if the combined additional parameter is below said predefined combined additional discriminating value.

Optionally, the aforementioned methods can include any one or more of the following steps: receiving a request to identify whether a patient sample from a gastrointestinal cancer patient contains markers that predict recurrence of gastrointestinal cancer; receiving a biological sample obtained from a preoperative gastrointestinal cancer patient; contacting a biological sample obtained from said selected patient with a first antibody to TIMP-1 or a probe that complements an mRNA encoding TIMP-1; contacting said biological sample obtained from said patient with a second antibody to CEA or a probe that complements an mRNA encoding CEA; determining a first value representing the amount of binding of said first antibody or the amount of TIMP-1 mRNA bound by the probe; determining a second value representing the amount of binding of said second antibody or the amount of CEA mRNA bound by the probe; inputting the first and second values into a computer configured to transform said first and second values into a prognostic index using an algorithm expressed by:

0.11×Log 2(CEA)+0.41×Log 2(TIMP-1)

determining whether said solved prognostic index is associated with recurrence of gastrointestinal cancer or remission of gastrointestinal cancer by comparing the solved prognostic index to a database containing a plurality of prognostic indices, wherein some of the indices are associated with a recurrence of gastrointestinal cancer and some of the indices are associated with remission of gastrointestinal cancer; and communicating said determination of whether said solved prognostic index is associated with recurrence of gastrointestinal cancer or remission of gastrointestinal cancer to said person making said request.

II. Methods for Determining Whether a Gastrointestinal Cancer Patient is Likely to Experience a Disease Recurrence or Disease Related Death Following Neo-Adjuvant or Adjuvant Anti-Cancer Therapy.

Another aspect of the present invention relates to a method for determining whether a gastrointestinal cancer patient is likely to experience a disease recurrence or disease related death following neo-adjuvant or adjuvant anti-cancer therapy, said method comprising
a) determining at a first time point the total concentration of TIMP-1 in a pre-operative plasma sample of said patient,
b) determining at said first time point the total concentration of CEA in a pre-operative plasma sample of said patient,
b) combining the concentration of TIMP-1 with the concentration of CEA to result in a combined TIMP-1/CEA parameter,
c) indicating the patient as likely to experience a disease recurrence or disease related death if the combined TIMP-1/CEA parameter is at or above a predefined combined TIMP-1/CEA discriminating value, and
d) indicating the individual as unlikely to experience a disease recurrence or disease related death if the combined TIMP-1/CEA parameter is below said predefined TIMP-1/CEA combined discriminating value,
e) if the individual is likely to experience a disease recurrence or disease related death determining at a second time point the total concentration of TIMP-1 in a post-operative plasma sample of said patient,
f) determining at said second time point the total concentration of CEA in a post-operative plasma sample of said patient,
g) combining the concentration of TIMP-1 with the concentration of CEA to result in a combined TIMP-1/CEA parameter,
h) indicating the patient as likely to experience a disease recurrence or disease related death if the combined TIMP-1/CEA parameter is at or above a predefined combined TIMP-1/CEA discriminating value, and
i) indicating the individual as unlikely to experience a disease recurrence or disease related death if the combined TIMP-1/CEA parameter is below said predefined TIMP-1/CEA combined discriminating value.

In a further embodiment the present invention pertains to a method for determining whether a gastrointestinal cancer patient is likely to experience a disease recurrence or disease related death following neo-adjuvant or adjuvant anti-cancer therapy, said method comprising
a) determining at a first time point the total concentration of TIMP-1 in a pre-operative plasma sample of said patient,
b) determining at said first time point the concentration of CEA in a pre-operative body fluid sample of said patient,
c) determining at said first time point at least one additional parameter pre-operatively of said patient,
d) combining the concentration of TIMP-1 with the concentration of CEA and the at least one additional parameter to result in a combined additional parameter,
e) indicating the patient as likely to experience a disease recurrence if the combined additional parameter is at or above a predefined combined additional discriminating value,
f) indicating the individual as unlikely to experience a disease recurrence or disease related death if the combined additional parameter is below said predefined combined additional discriminating value,
g) if the individual is likely to experience a disease recurrence or disease related death determining at a second time point the total concentration of TIMP-1 in a post-operative plasma sample of said patient,
h) determining at said second time point the concentration of CEA in a post-operative body fluid sample of said patient,
i) determining at said second time point at least one additional parameter post-operatively of said patient,
j) combining the concentration of TIMP-1 with the concentration of CEA and the at least one additional parameter to result in a combined additional parameter,
k) indicating the patient as likely to experience a disease recurrence if the combined additional parameter is at or above a predefined combined additional discriminating value, and
l) indicating the individual as unlikely to experience a disease recurrence or disease related death if the combined additional parameter is below said predefined combined additional discriminating value.

Thus, in one embodiment the present invention pertains to the determination of changes in the concentrations of plasma levels of TIMP-1 and serum or plasma levels of CEA and optionally at least one additional parameter (e.g., the prognostic index solved by formula A) between a first time point and a second time point in the course of a patient's gastrointestinal cancer disease. By measuring the changes in the concentrations of plasma levels of TIMP-1 and serum or plasma levels of CEA and optionally at least one additional parameter (e.g., the prognostic index solved by formula A) between a first time point and a second time point in the course of a patient's gastrointestinal cancer disease one can determine whether a patient is likely to experience a disease recurrence or a disease related death. The measurement may also be performed between a first and third or further time point or between a second and third or further time point. Such determination can be performed using any of the above disclosed methods—i.e. the methods for determining whether a gastrointestinal cancer patient is likely to experience a disease recurrence or disease related death following neo-adjuvant or adjuvant anti-cancer therapy.

In one embodiment the present invention pertains to a method for determining whether a gastrointestinal cancer patient is likely to experience a disease recurrence or disease related death following neo-adjuvant or adjuvant anti-cancer therapy. This method comprises determining at a first time point the total concentration of TIMP-1 in a pre-operative plasma sample and determining at said first time point the concentration of CEA in a pre-operative body fluid sample both samples obtained from patient, and additionally combining the measured concentration of TIMP-1 with the measured concentration of CEA to result in a combined TIMP-1/CEA parameter. If the combined TIMP-1/CEA parameter is at or above a predefined combined TIMP-1/CEA discriminating value it indicates that the patient is likely to experience a disease recurrence or disease related death. If the combined TIMP-1/CEA parameter is below said predefined TIMP-1/CEA combined discriminating value it indicates that the individual is unlikely to experience a disease recurrence or disease related death.

If the patient is likely to experience a disease recurrence or disease related death the patient is preferably treated with a form of therapy (e.g., surgery or neo-adjuvant therapy). The method further comprising determining at a second time point the total concentration of TIMP-1 in a post-operative plasma sample and determining at said second time point the concentration of CEA in a post-operative body fluid sample both samples obtained from patient, and additionally combining the measured concentration of TIMP-1 with the measured concentration of CEA to result in a combined TIMP-1/CEA parameter. If the combined TIMP-1/CEA parameter is at or above a predefined combined TIMP-1/CEA discriminating value it indicates that the patient is likely to experience a disease recurrence or disease related death. If the combined TIMP-1/CEA parameter is below said predefined TIMP-1/CEA combined discriminating value it indicates that the individual is unlikely to experience a disease recurrence or disease related death.

If the patient is unlikely to experience a disease recurrence or disease related death and the second time point is during treatment the treatment is continued. If the patient is unlikely to experience a disease recurrence or disease related death and the second time point is after termination of the treatment the patient is preferably not treated any further with said treatment. If on the other hand the patient is likely to experience a disease recurrence or a disease related death the patent is preferably switched to another treatment. In one embodiment said treatment is a more aggressive treatment.

The above also applies where the at least one additional parameter is measured and included in the method (see e.g. claim 4).

In one embodiment the first time point is pre-operatively. The first time point may be selected from the group consisting of 1, 2, 3, 4, 5, 6, 7, 8, 9, 10, 11, 12, 13, 14, 15, 16, 17, 18, 19, 20, 21, 22, 23 or 24 months before initiation of the treatment or before the primary treatment (e.g. surgery) e.g. in the range of 1-24 months before initiation of the treatment or before the primary treatment, such as in the range of 2-23 months, e.g. in the range of 3-22 months, such as in the range of 4-21 months, e.g. in the range of 5-20 months, such as in the range of 6-19 months, e.g. in the range of 7-18 months, such as in the range of 8-17 months, e.g. in the range of 9-16 months, such as in the range of 10-15 months, e.g. in the range of 11-14 months, such as in the range of 12-13 months, e.g. in the range from 1-5 months, such as 2-6 months. Time points less than or equal to 1 month include but is not limited to 1 week, 2 weeks or 3 weeks. In one embodiment the first time point may be selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 and 31 days before initiation of the treatment or before the primary treatment (e.g. surgery).

In one embodiment the second time point is post-operatively. The second time point may be during treatment and/or after termination of the treatment. The second time point may be selected from the group consisting of 1, 2, 3, 4, 5, 6, 7, 8, 9, 10, 11, 12, 13, 14, 15, 16, 17, 18, 19, 20, 21, 22, 23 or 24 months after initiation the of the treatment or termination of the treatment e.g. in the range of 1-24 months after initiation the of the treatment or termination of the treatment, such as in the range of 2-23 months, e.g. in the range of 3-22 months, such as in the range of 4-21 months, e.g. in the range of 5-20 months, such as in the range of 6-19 months, e.g. in the range of 7-18 months, such as in the range of 8-17 months, e.g. in the range of 9-16 months, such as in the range of 10-15 months, e.g. in the range of 11-14 months, such as in the range of 12-13 months, e.g. in the range from 1-5 months, such as 2-6 months. Time points less than or equal to 1 month include but is not limited to 1 week, 2 weeks or 3 weeks. In one embodiment the second time point may be selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 and 31 days after initiation of the treatment and/or termination of the treatment.

Thus the present invention pertains to the determination of the biomarkers (e.g. TIMP-1 and CEA and optionally at least one additional parameter) following neo-adjuvant and/or adjuvant therapy, thereby obtaining information on the efficacy of the given neo-adjuvant or adjuvant treatment. I.e. the invention pertains to a method for determining whether a gastrointestinal cancer patient is likely to experience a disease recurrence or disease related death following or during neo-adjuvant or adjuvant anti-cancer therapy.

In the case of information that indicates lack of efficacy of the given neo-adjuvant and/or adjuvant treatment (i.e. the patient is likely to experience a disease recurrence or a disease related death), the patients should be offered a second course of neo-adjuvant and/or adjuvant treatment with anti-cancer treatment modalities not exhibiting cross resistance patterns with the anti-cancer treatment modalities that were administered as first line treatment.

In the case of information that indicates efficacy of the given neo-adjuvant and/or adjuvant treatment (i.e. the patient is unlikely to experience a disease recurrence or a disease related death), the patients should be offered continued course of neo-adjuvant and/or adjuvant treatment (i.e. the first line treatment). Alternatively the treatment should be stopped if the medical practitioner finds the patient is cured for the gastrointestinal cancer disease.

Such information on the efficacy of neo-adjuvant or adjuvant treatment can in accordance with the present invention be obtained by measuring the biomarker levels at more than one time point (i.e. a first and a second time point) in the course of the disease, the second time point may be during treatment or months after the initiation or termination of treatment. When comparing biomarker levels between time points (as disclosed in the methods for determining whether a gastrointestinal cancer patient is likely to experience a disease recurrence or disease related death following neo-adjuvant or adjuvant anti-cancer therapy) a risk assessment for lack of therapy efficacy of the given therapy can be estimated.

Optionally, the aforementioned methods can include any one or more of the following steps: receiving a request to identify whether a patient sample from a gastrointestinal cancer patient contains markers that predict recurrence of gastrointestinal cancer; receiving a biological sample obtained from a preoperative gastrointestinal cancer patient; contacting a biological sample obtained from said selected patient with a first antibody to TIMP-1 or a probe that complements an mRNA encoding TIMP-1; contacting said biological sample obtained from said patient with a second antibody to CEA or a probe that complements an mRNA encoding CEA; determining a first value representing the amount of binding of said first antibody or the amount of TIMP-1 mRNA bound by the probe; determining a second value representing the amount of binding of said second antibody or the amount of CEA mRNA bound by the probe; inputting the first and second values into a computer configured to transform said first and second values into a prognostic index using an algorithm expressed by:

0.11×Log 2(CEA)+0.41×Log 2(TIMP-1)

determining whether said solved prognostic index is associated with recurrence of gastrointestinal cancer or remission of gastrointestinal cancer by comparing the solved prognostic index to a database containing a plurality of prognostic indices, wherein some of the indices are associated with a recurrence of gastrointestinal cancer and some of the indices are associated with remission of gastrointestinal cancer; and communicating said determination of whether said solved prognostic index is associated with recurrence of gastrointestinal cancer or remission of gastrointestinal cancer to said person making said request.

In some embodiments the combined parameters are used to generate a risk assessment score exhibiting indicia of risk of disease recurrence or death of the cancer patient based on the collective value of the biomarkers; TIMP-1, CEA and optionally at least one additional tumour marker.

In one embodiment the additional tumour marker is selected from the group consisting of serum or plasma soluble suPAR, serum or plasma CA19.9, serum or plasma CA246, un-complexed TIMP-1, TIMP-1 in complex with specific MMP's, soluble CD63, YKL-40, p66 Shc, MMP's, ADAM's and kallekreins and combinations thereof.

A description on how to determine the discriminating value can be found in the section termed “Discriminating values”.

Some embodiments of the present invention also include methods of determining whether a subject has a poor prognosis for a gastrointestinal cancer disease, i.e. shorter time to disease recurrence or disease related death. By some approaches, these methods comprise measuring the level of a panel of biomarkers in a biological sample from the cancer patient, wherein the result of the measurements are indicative of the patient having a high or low proclivity for disease. Again, many of these methods may include any one or more of the following steps: receiving a request to identify whether a patient sample from a gastrointestinal cancer patient contains markers that predict recurrence of gastrointestinal cancer; receiving a biological sample obtained from a preoperative gastrointestinal cancer patient; contacting a biological sample obtained from said selected patient with a first antibody to TIMP-1 or a probe that complements an mRNA encoding TIMP-1; contacting said biological sample obtained from said patient with a second antibody to CEA or a probe that complements an mRNA encoding CEA; determining a first value representing the amount of binding of said first antibody or the amount of TIMP-1 mRNA bound by the probe; determining a second value representing the amount of binding of said second antibody or the amount of CEA mRNA bound by the probe; inputting the first and second values into a computer configured to transform said first and second values into a prognostic index using an algorithm expressed by:

0.11×Log 2(CEA)+0.41×Log 2(TIMP-1)

determining whether said solved prognostic index is associated with recurrence of gastrointestinal cancer or remission of gastrointestinal cancer by comparing the solved prognostic index to a database containing a plurality of prognostic indices, wherein some of the indices are associated with a recurrence of gastrointestinal cancer and some of the indices are associated with remission of gastrointestinal cancer; and communicating said determination of whether said solved prognostic index is associated with recurrence of gastrointestinal cancer or remission of gastrointestinal cancer to said person making said request.

Gastrointestinal Cancer

Gastrointestinal cancer refers to malignant conditions of the gastrointestinal tract, including but not limited to the esophagus, stomach, liver, biliary system, pancreas, bowels, rectum and anus. The term gastrointestinal cancer is a genus thus covering several species such as but not limited to oesophageal cancers, gastric cancers, small intestine cancers, gall bladder cancers, liver cancers, pancreatic cancers and colorectal cancers such as colon cancers and rectal cancers.

Accordingly, in one embodiment of the invention the gastro-intestinal cancer is selected from the group consisting of oesophageal cancers, gastric cancers, small intestine cancers, gall bladder cancers, liver cancers, pancreatic cancers and colorectal cancers such as colon cancers and rectal cancers.

In a second embodiment, the gastrointestinal cancer is an adenocarcinoma selected from the group consisting of colon adenocarcinomas and rectal adenocarcinomas.

In a preferred embodiment, the gastro-intestinal cancer is colorectal cancer.

Colorectal Cancer (CRC)

In the present context the term “colorectal cancer” (CRC) is to be understood as a species of the genus “gastrointestinal cancer”.

CRC according to the invention refers to cancerous growths in the colon, rectum and appendix.

Stage II corresponds to the Dukes B class of the Dukes classification system for colorectal cancer. Accordingly, Stage III corresponds to the Dukes C class of the Dukes classification system for colorectal cancer.

The terms colorectal cancer, colon cancer, large bowel cancer and CRC are used herein interchangeably.

In a further embodiment, said colorectal cancer is selected from the group consisting of stage II colorectal cancer and stage III colorectal cancer.

Adjuvant or Neo-Adjuvant Anti-Cancer Treatment (Adjuvant or Neo-Adjuvant Anti-Cancer Therapy)

Adjuvant anti-cancer treatment refers to additional treatment, such as additional systemic treatment provided after surgery where all detectable tumour tissue has been removed, but where there remains a statistical risk of relapse due to occult disease.

Adjuvant anti-cancer treatment is provided soon after the primary surgical removal of the tumour tissue (i.e. the primary treatment).

Accordingly, in one embodiment of the present invention the adjuvant anti-cancer treatment is provided less than 8 weeks following the primary treatment, such less than 7 weeks post-primary treatment, for example less than 6 weeks post-primary treatment, such less than 5 weeks post-primary treatment, for example less than 4 weeks post-primary treatment, such less than 3 weeks post-primary treatment, for example less than 2 weeks post-primary treatment, such less than 1 weeks post-primary treatment.

In a particular embodiment, the adjuvant anti-cancer treatment is provided 3 to 4 weeks following the primary treatment.

Neo-adjuvant therapy on the other hand is provided before the primary treatment such as chemotherapy that is given before surgical removal of a tumour in the gastrointestinal tract. The most common reason for neo-adjuvant therapy is to reduce the size of the tumour so as to facilitate more effective surgery removal.

Accordingly, in one embodiment of the present invention the neo-adjuvant anti-cancer treatment is provided less than 8 weeks before the primary treatment, such less than 7 weeks before the primary treatment, for example less than 6 weeks before the primary treatment, such less than 5 weeks before the primary treatment, for example less than 4 weeks before the primary treatment, such less than 3 weeks before the primary treatment, for example less than 2 weeks before the primary treatment, such less than 1 weeks before the primary treatment.

In one embodiment the anti-cancer treatment is selected from the group consisting of neo-adjuvant therapy, adjuvant therapy, and therapy of metastatic disease.

In a particular embodiment the adjuvant treatment is a systemic anti-cancer treatment.

In one embodiment according to the invention, the adjuvant chemotherapy following primary surgical removal of the tumour is selected from the group consisting of FOLFOX (infusional 5-fluorouracil, leucovorin, and oxaliplatin), 5-fluorouracil (5-FU), Capecitabine (Xeloda), Leucovorin (LV, Folinic Acid) and Oxaliplatin (Eloxatin) or consisting of FOLFIRI (infusional 5-fluorouracil, leucovorin, and irinotecan).

In another embodiment the adjuvant chemotherapy is selected from the group consisting of the combination of FOLFOX with bevacizumab, FOLFIRI (infusional 5-fluorouracil, leucovorin, and irinotecan) with bevacizumab, 5-fluorouracil (5-FU), Capecitabine, Leucovorin (LV, Folinic Acid), Irinotecan (Camptosar), Oxaliplatin (Eloxatin), Bevacizumab (Avastin), Cetuximab (Erbitux), and Panitumumab (Vectibix).

Tissue Inhibitor of Metalloprotease-1 (TIMP-1)

TIMP-1 is one out a family of four endogenous inhibitors of matrix metalloproteases (MMPs). TIMP-1 is a 28 kDa protein which binds most MMPs with a 1:1 stochiometry. TIMP-1 is present in various tissues and body fluids and is stored in α-granules of platelets and released upon activation. While the main function of TIMP-1 is supposed to be MMP inhibition, some alternative functions of TIMP-1 have been described, e.g. inhibition of apoptosis and regulation of cell growth and angiogenesis. In addition, some studies have suggested that TIMP-1 may also play a role in the early processes leading to the malignant phenotype. TIMP-1 exists both in free form and in the form of complexes with metalloproteinases, and it has been found that an important parameter is the total concentration of TIMP-1, that is, the sum of the TIMP-1 in free form and the TIMP-1 in complex forms. Free or uncomplexed TIMP-1 is found in plasma and serum of healthy individuals as well as in plasma and serum of cancer patients (Holten-Andersen et al., Clin Chem 2004). The inventors have developed a specific ELISA, which detects total TIMP-1 or uncomplexed TIMP-1 with high sensitivity in a blood sample Holten-Andersen et al., Br J Cancer 1999; Holten-Andersen et al., Clin Chem 2004).

The parameter representing the concentration of TIMP-1 may be the concentration proper of TIMP-1. It will be understood that the other expressions than the concentration proper can represent the concentration, such as, e.g., the concentration multiplied by a factor, etc., and that such other representations can be used equally well for the purpose of the present invention provided the corresponding adjustments are made.

According to the present invention TIMP-1 level may be determined either by quantitative determination of the TIMP-1 mRNA of the sample in question or by determining the TIMP-1 protein concentration of said sample.

In one embodiment the TIMP-1 mRNA concentration of the sample in question correlates with TIMP-1 protein concentration of said sample. Accordingly, TIMP-1 mRNA concentration reflects the TIMP-1 protein concentration of said sample.

Thus, in one embodiment of the present invention the TIMP-1 concentration is selected from the group consisting of the TIMP-1 mRNA concentration of said sample and TIMP-1 protein concentration of said sample.

In another embodiment of the present invention the TIMP-1 mRNA concentration is measured in one type of sample while the TIMP-1 protein determination is performed in another type of sample.

In one embodiment according to the invention the concentration of TIMP-1 is selected from the group consisting of the concentration of TIMP-1 in free form, the concentration of TIMP-1 in complex form, and the total concentration of TIMP-1.

In a second embodiment the total concentration of TIMP-1 is the concentration of free TIMP-1 and TIMP-1 in complex form.

In a particular embodiment the TIMP-1 protein concentration is the TIMP-1 protein concentration of said plasma sample.

Carcinoembryonic Carcinogen (CEA)

Carcinoembryonic carcinogen (CEA) is a glycoprotein involved in cell adhesion. CEA is expressed during fetal development and down-regulated before birth. CEA is the only recommended serological biomarker to be used to guide treatment of colorectal cancer patients. CEA is approved for monitoring of colorectal cancer patients, allowing for early therapeutic intervention in the case of a rise in serum CEA levels in a patient with prior or current colorectal cancer.

According to the present invention CEA level may be determined either by quantitative determination of the CEA mRNA of the sample in question or by determining the CEA protein concentration (e.g., by ELISA of said sample or another type of sample).

In one embodiment the CEA mRNA concentration of the sample in question correlates with CEA protein concentration of said sample. Accordingly, CEA mRNA concentration reflects the CEA protein concentration of said sample.

Thus, in one embodiment according to the present invention said CEA concentration is selected from the group consisting of the CEA mRNA concentration of said body fluid sample and CEA protein concentration of said body fluid sample or another type of sample.

In another embodiment the CEA concentration is selected from the group of the serum CEA concentration of said body fluid sample, the plasma CEA concentration of said body fluid sample, and the CEA concentration in plasma and serum of said body fluid sample. CEA concentration can also be determined by evaluating the amount of antibody bound to CEA in a sample (e.g., by immunoassays such as ELISA).

Determination of the TIMP-1, CEA and additional tumour marker concentration respectively, may in some embodiment allow the use of the same sample. In other embodiment, TIMP-1, CEA and additional tumour marker concentration respectively are performed on individual samples obtained from said patient.

In a particular embodiment of the present invention, the concentration of TIMP-1 and the CEA concentration are determined using the same sample.

Additional Parameter

The methods according to the invention may include additional parameters to take into account other available data, which is relevant for the clinical outcome.

One aspect of the present invention concerns methods for determining whether a gastrointestinal cancer patient is likely to experience a disease recurrence or disease related death, said method comprising
a) determining the total concentration of TIMP-1 in a pre-operative plasma sample of said patient, and
b) determining the concentration of CEA in a pre-operative body fluid sample of said patient, and
c) determining at least one additional parameter pre-operatively of said patient, and
d) combining the concentration of TIMP-1 with the concentration of CEA and the at least one additional parameter to result in a combined additional parameter
e) indicating the patient as likely to experience a disease recurrence or disease related death if the combined additional parameter is at or above a predefined combined additional discriminating value, and
f) indicating the individual as unlikely to experience a disease recurrence or disease related death if the combined additional parameter is below said predefined combined additional discriminating value.

Yet another aspect of the present invention concerns a method for determining whether a gastrointestinal cancer patient is likely to experience a disease recurrence or disease related death following neo-adjuvant or adjuvant anti-cancer therapy, said method comprising
a) determining at a first time point the total concentration of TIMP-1 in a pre-operative plasma sample of said patient,
b) determining at said first time point the concentration of CEA in a pre-operative body fluid sample of said patient,
c) determining at said first time point at least one additional parameter pre-operatively of said patient,
d) combining the concentration of TIMP-1 with the concentration of CEA and the at least one additional parameter to result in a combined additional parameter,
e) indicating the patient as likely to experience a disease recurrence if the combined additional parameter is at or above a predefined combined additional discriminating value,
f) indicating the individual as unlikely to experience a disease recurrence or disease related death if the combined additional parameter is below said predefined combined additional discriminating value,
g) if the individual is likely to experience a disease recurrence or disease related death determining at a second time point the total concentration of TIMP-1 in a post-operative plasma sample of said patient,
h) determining at said second time point the concentration of CEA in a post-operative body fluid sample of said patient,
i) determining at said second time point at least one additional parameter post-operatively of said patient,
j) combining the concentration of TIMP-1 with the concentration of CEA and the at least one additional parameter to result in a combined additional parameter,
k) indicating the patient as likely to experience a disease recurrence if the combined additional parameter is at or above a predefined combined additional discriminating value, and
l) indicating the individual as unlikely to experience a disease recurrence or disease related death if the combined additional parameter is below said predefined combined additional discriminating value.

In one embodiment of the present invention, the at least one additional parameter is selected from the group consisting of age, gender, general clinical status, tumour localization, stage of disease, differentiation grade, lymphatic, vascular vessel invasion, nerve invasion, co-morbidity, adjuvant treatment regime and an additional tumour marker different from any form of TIMP-1 or CEA, in a body fluid sample from the individual.

In a particular embodiment of the present invention, the at least one additional parameter is selected from the group consisting of age, gender, general clinical status, tumour localization, stage of disease, differentiation grade, lymphatic, vascular vessel invasion, nerve invasion, co-morbidity, adjuvant treatment regime and an additional tumour marker different from any form of TIMP-1 or CEA, in a body fluid sample from the patient.

According to the present invention, the additional parameter may be a tumour marker relevant for the gastrointestinal cancer in question, where said additional tumour marker is not TIMP-1 or CEA.

In one embodiment according to the invention the additional tumour marker is selected from the group consisting of serum or plasma soluble suPAR, serum or plasma CA19.9, serum or plasma CA246, un-complexed TIMP-1, TIMP-1 in complex with specific MMP's, soluble CD63, YKL-40, p66 Shc, MMP's, ADAM's and kallekreins and combinations thereof.

Optionally, the aforementioned methods can include any one or more of the following steps: receiving a request to identify whether a patient sample from a gastrointestinal cancer patient contains markers that predict recurrence of gastrointestinal cancer; receiving a biological sample obtained from a preoperative gastrointestinal cancer patient; contacting a biological sample obtained from said selected patient with a first antibody to TIMP-1 or a probe that complements an mRNA encoding TIMP-1; contacting said biological sample obtained from said patient with a second antibody to CEA or a probe that complements an mRNA encoding CEA; determining a first value representing the amount of binding of said first antibody or the amount of TIMP-1 mRNA bound by the probe; determining a second value representing the amount of binding of said second antibody or the amount of CEA mRNA bound by the probe; inputting the first and second values into a computer configured to transform said first and second values into a prognostic index using an algorithm expressed by:

0.11×Log 2(CEA)+0.41×Log 2(TIMP-1)+WW×Log 2(additional parameter)

WW is determined based on the regression analysis performed, and if the regression is a linear regression (e.g.) correspond to the slope of the line. In this sense, WW may be considered as the concentration or status of the additional parameter.

Determining whether said solved prognostic index is associated with recurrence of gastrointestinal cancer or remission of gastrointestinal cancer by comparing the solved prognostic index to a database containing a plurality of prognostic indices, wherein some of the indices are associated with a recurrence of gastrointestinal cancer and some of the indices are associated with remission of gastrointestinal cancer; and communicating said determination of whether said solved prognostic index is associated with recurrence of gastrointestinal cancer or remission of gastrointestinal cancer to said person making said request.

Mathematical Methods

The combined parameter (e.g., the combined C_{TIMP-1}/C_{CEA} parameter and the combined additional parameter) may be generated using any suitable statistical method(s) such as logistic regression analysis—or as will be disclosed in the following by mathematical operations in general, assembling an algorithm. While the underlying data (data upon which the mathematical operations are performed) express states in a population, statistical methods are often applied to data to provide what is called a discriminating value and a prognostic indicator (PI). The discriminating value is derived from a population with known course of disease where PI is determined for a patient in question at a particular disease state. Thus, in some embodiments the combining step is performed by logistic regression analysis although other methods are within the reach of the present invention.

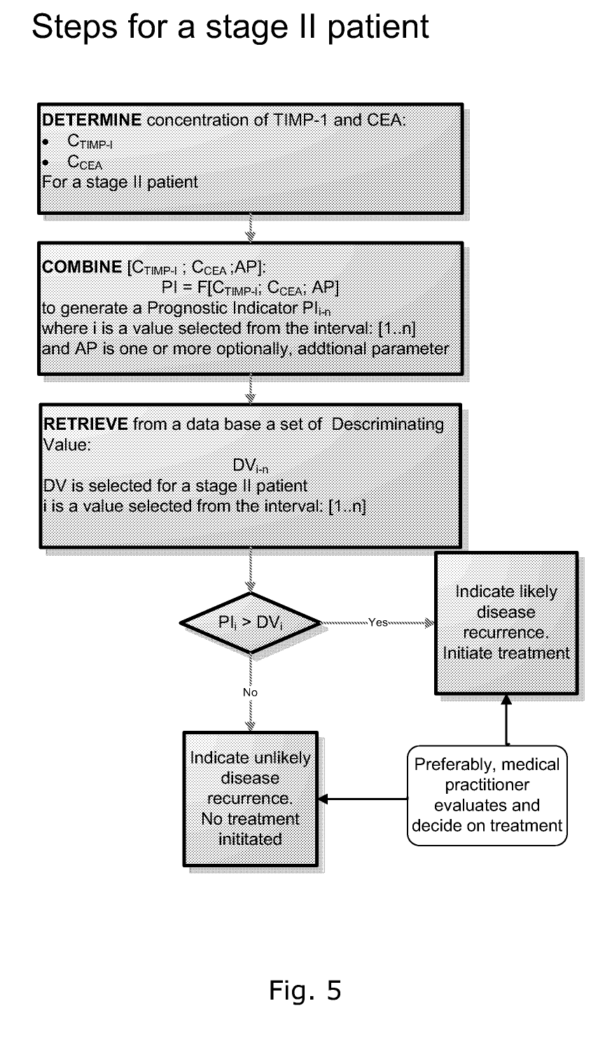

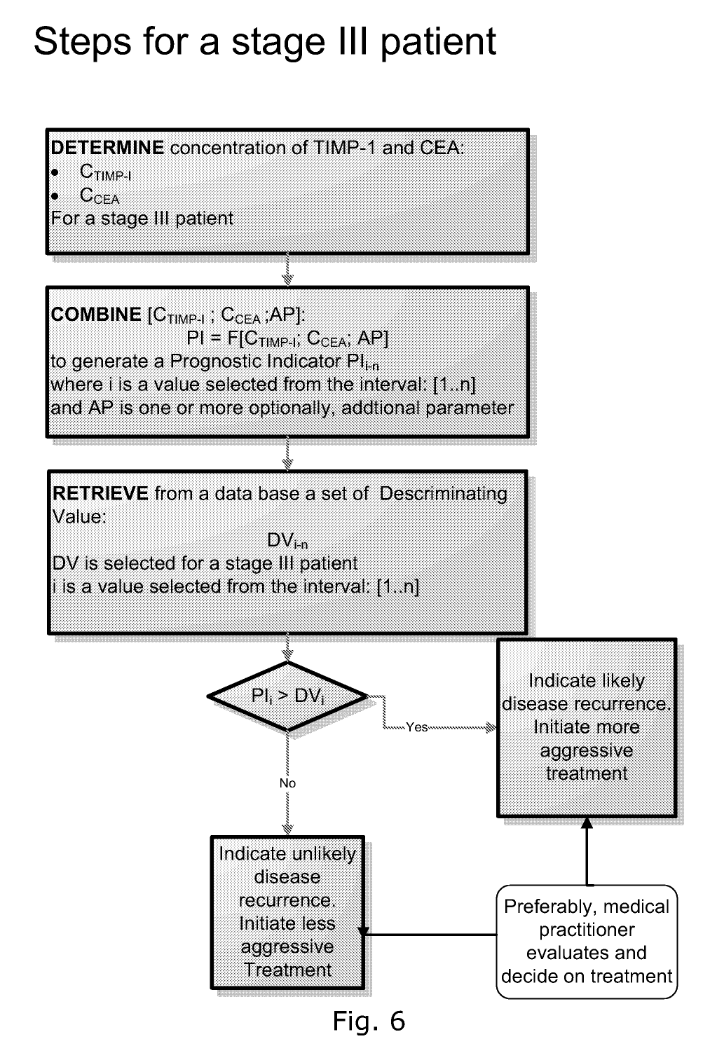

FIGS. 5 and 6 disclose the overall, preferred steps in processes according to preferred embodiments of the present invention to be carried out in order to reach an indication on whether it is likely that a given patient (in FIG. 5 the patient is at stage II and in FIG. 6, the patient is at stage III) will experience disease recurrence if the treatment scheme the patient in question follows is maintained. The indication is provided by comparing the Prognostic Indicator, PI, with a Discriminating Value to provide estimate on whether a patient is statistically likely to experience a disease recurrence or not. As further indicated in FIGS. 5 and 6 a medical practitioner evaluates and decides on what to do next so that the suggestions as to treatment etc still is in the responsibility of the medical practitioner.

As it appears from the flow chart shown in FIGS. 5 and 6, the process is initiated by determining the concentration of TIMP-1 and CEA, i.e. C_{TIMP-1} and C_{CEA}. Once these concentrations are determined, they are to be algebraically combined by mathematical operation (as will be disclosed in further details below for preferred embodiments) and many such algebraic combinations are within the reach of the present invention. Accordingly, the invention may preferably and advantageously be disclosed by using the symbol F for a mathematical operator providing algebraic combination assembling an algorithm. The mathematical operator F may preferably comprise a number of basic mathematical operations like “+”, “−”, “/” and “*”, and in a particular preferred embodiment the mathematical operator F comprising

PI=F[C_{CEA};C_{TIMP-1}]

where

F[PAR_{—}1;PAR_{—}2]=0.11 Log₂(PAR_{—}1)+0.41 Log₂(PAR_{—}2)

is the algorithm corresponding to F in a particular preferred embodiment. The first parameter PAR_{—}1 is C_{CEA} and the second parameter PAR_{—}2 is C_{TIMP-1}, so that

PI=0.11 Log₂(C_{CEA})+0.41 Log₂(C_{TIMP-1})

As shown above, an additional parameter, AP, may be taken into account when estimating PI so that PI=F[C_{CEA}; C_{TIMP-1}; AP]. In such cases, F may preferably be in the following form:

F[PAR_{—}1;PAR_{—}2,AP]=0.11 Log₂(PAR_{—}1)+0.41 Log₂(PAR_{—}2)+WW Log₂(AP)

is the algorithm corresponding to F in another preferred embodiment. WW is the concentration or status of the additional parameter (as disclosed above).

In a further embodiment F[PAR_{—}1; PAR_{—}2]=PAR_{—}1/Par_{—}2 so that PI=C_{TIMP-1}/C_{CEA}.

Furthermore, PI may be a single value or maybe a set of values. This is indicated by giving PI an index “i”. e.g. PIᵢ where i is selected from the interval [1 . . . n]. In the example above, n=1 and the index is omitted. In other cases n equals 2 and the prognostic indicator is accordingly a set of values like PI₁ and PI₂.

In the two above identified cases, the comparison is preferably performed in the following manner:
IS PV≧DV? (answer=“yes” or “no”)
IF yes THEN “Indicate likely disease recurrence”
IF no THEN “Indicate unlikely disease recurrence”

The Prognostic Indicator, PI, determined is to be compared with a Discriminating Value, DV. As in the case with the Prognostic Indicator, PI, the Discriminating Value may either be a single value or a set of values and the same nomenclature (subscript) is used for DV as for PI.

An embodiment where PI and DV are sets of values comprises

PI_{1,2}=F[C_{CEA};C_{TIMP-1}]->PI₁=C_{CEA},PI₂=C_{TIMP-1}

In this case, DV is accordingly,

DV_{1,2}=[DV₁;DV₂]

which are retrieved from a database. In this case, the comparison is performed by
PI₁≧DV₁ AND PI₂≧DV₂ (answer=“yes” or “no”)
IF yes THEN “Indicate likely disease recurrence”
IF no THEN “Indicate unlikely disease recurrence”

Commonly for the Discriminating Values is that they are stored in a database and the DVs are available for stage II and stage III patients. Furthermore, the DVs stored preferably correspond to a particular mathematical operator F. The DVs are preferably stored with a tag indicating whether a particular DV is for stage II or stage III patient and the particular mathematical operator to which it correspond as the PI and the DV compared should preferably match each other in the sense that if:
PI is determined for a stage II patient with a particular mathematical operator F, then the DV to be compared with should preferably also be for
   a stage II patient and
   the particular mathematical operator used for obtaining PI
PI is determined for at stage III patient with a particular mathematical operator F, then the DV to be compared with should preferably also be for
   a stage III patient and
   the particular mathematical operator used for obtaining PI

Accordingly, DVs for at given set of patients are calculated by a mathematical operator as outlined above based on determinations of e.g. C_{TIMP-1} and C_{CEA}. This is done based on data obtained from retrospective studies in which we know the fait of the disease in each individual patient and in whom we do have C_{TIMP-1} and

C_{CEA} values obtained preoperatively (see description of discriminating value below)

The method is executed by use of a computer comprising storage means and CPU adapted to perform at least the combination step and the retrieving step. Preferably, the comparing step is also performed by the computer. Accordingly, the data base storing DVs are stored in a computer system so that the CPU can access the DVs.

Methods of Sample Analysis

The determination of TIMP-1, CEA, and optionally the at least one additional parameter may be performed using any suitable methods known in the art, for example protein may be determined using ELISA, RIA, immunohistochemistry, Western blotting, mass spectroscopy, flow cytometry, antibody mediated pull-down assay, and antibody arrays.

Other methods of analysis suitable for determining RNA such mRNA in a samples comprise methods of PCR, methods of RT-PCR such as qRT-PCR, and microarrays suitable for RNA analysis.

In one embodiment according to the invention the determination of TIMP-1 is performed by means of an immunoassay selected from the group consisting of ELISA, RIA, immunohistochemistry, Western blotting, mass spectroscopy, flow cytometry, antibody mediated pull-down assay.

In second embodiments according to the invention the determination of CEA is performed by means of an immunoassay selected from the group consisting of ELISA, RIA, immunohistochemistry, Western blotting, mass spectroscopy, flow cytometry, antibody mediated pull-down assay.

In third embodiments according to the invention the determination the concentration of TIMP-1 is determined using a method selected from the group consisting of a histological method, a cytological method, a method of PCR, a method of RT-PCR such as qRT-PCR.

In fourth embodiments according to the invention the determination of the concentration of CEA is determined using a method selected from the group consisting of a histological method, a cytological method, a method of PCR, a method of RT-PCR such as qRT-PCR and a method for the detection of CEA gene aberrations, e.g. CEA gene deletions or amplifications.

The reagents used to detect the protein or RNA in question may be labelled, radio-labelled, fluorescence labelled or biotin labelled. Antibodies used for detecting the protein in question may radio-labelled, chromophore-labelled, fluorophore labelled or enzyme labelled.

Samples (Biological Sample)

Blood sample according to the present invention refers to a blood sample comprising whole blood, blood plasma and blood serum.

A sample (biological samples) also refers to a body fluid sample such but not limited to plasma, serum, urine, faeces and saliva.

A sample (biological sample) according to the invention further comprises tumor tissue and tumor tissue comprising non-malignant tissue.

Discriminating Values

The discriminating value is a value which has been determined by measuring the parameter in a cohort of CRC patients with full follow-up data, e.g. information on time to any CRC disease recurrence or CRC related death and then identifying the biomarker discriminating value which gives the most optimal discrimination between CRC patients who do not develop disease recurrence or disease related death within 5 years of follow-up after the primary surgical treatment and patients who either develop disease recurrence or experience disease related death within 5 years of the primary treatment for CRC. The discriminating value of the biomarkers in question or the common algorithm (formula A), can also be established based on a predetermined specificity or a predetermined sensitivity based on an analysis of the relation between the parameter values and the known clinical data of the CRC patients in the studied cohort. The discriminating value determined in this manner is valid for the same experimental setup in future individual tests.

The discrimination value can be calculated based on information obtained from a retrospectively collected patient material e.g. plasma samples obtained pre-operatively from colorectal cancer patients and stored in the frozen state. These samples can at any time following the primary treatment of the patients be thawn and analyzed for biomarker (e.g. TIMP-1 and CEA) content. The results of these analyses can then be compared to the clinical information, e.g. time to disease recurrence and death, stored for each patient. Several statistical methods can now be applied to define the discrimination value i.e. the biomarker value which gives the best separation between those patients with a low likelihood of disease recurrence and those patients with a high likelihood of disease recurrence.

When the discrimination value has been defined this value can now be tested in a similar but independent retrospective sample material as described above. When confirmed the discriminating value is ready for use in routine patient management. The identification of the discriminating value should be performed for each of the two stages—i.e. stage II and stage III—since the value might not be the same.

The described procedure for identifying and validating the discriminating value of a prognostic biomarker (e.g. TIMP-1 and CEA) is in accordance with the current guidelines for FDA approval and has recently been used in several other prognostic biomarker studies (Bogaert et al.).

In one specific experimental setups, the concentration threshold of total TIMP-1 useful as a discriminating value was found to be in the range of 50-160 microgram/L of total TIMP-1 at a specificity of 90%. Other experimental setups and other parameters will result in other values which can be determined in accordance with the teachings herein.

In one embodiment according to the present invention the TIMP-1 discriminating value refers to the median plasma TIMP-1 concentration.

In another embodiment according to the present invention the TIMP-1 discriminating value is based on a cut point defined by at least or equal to 35%, 30%, 25%, 20%, or 15% of the highest TIMP-1 levels.

Using the same approach discriminating values are determined for CEA. That is, the CEA discriminating value is based on a cut point defined by at least or equal to 35%, 30%, 25%, 20%, or 15% of the highest CEA levels.

The discriminating value may be further adjusted according to other data available such as gender, age, co-morbidity, other clinic conditions, health in general. Other relevant demographic parameters and desire to divide the patients into pre-specified percentages, e.g. greater than or equal to 65%, 60%, 55%, 50%, 45%, 40% low (low risk) and at least 35%, 30%, 25%, 20%, 15%, 10% or 5% high (high risk). A discriminating value which is further adjusted includes the combined additional discriminating value.

Example 1 comprises prognostic index data—i.e. the prognosis for recurrence is determined in Example 1.

In the present context specificity is defined as the proportion of low risk patients who are correctly identified by the described method of the invention. In the present context sensitivity is defined as the proportion of high risk patients who are correctly identified by the described method.

In determining the discriminating value distinguishing individuals likely to experience a disease recurrence, the person skilled in the art has to predetermine the level of specificity. The ideal diagnostic test is a test that has 100% specificity, i.e. only detects individuals with recurrence and therefore no false positive results, and 100% sensitivity, i.e. detects all individuals with recurrence and therefore no false negative results. However, due to biological diversity no method can be expected to have 100% sensitive without including a substantial number of false negative results.

Accordingly, aspects of the invention relate to a method to divide stage II (Dukes B/lymph node negative) and stage III (Dukes C/lymph node positive) CRC patients into different prognostic groups. Patients with a poor prognostic profile based on the combined CEA and TIMP-1 levels in preoperatively obtained serum or plasma should be offered neo-adjuvant or adjuvant systemic anti cancer therapy independent of stage of disease. Stage III patients with a good prognostic profile based on the combined serum or plasma CEA and TIMP-1 levels could be offered less toxic neo-adjuvant or adjuvant therapy than the stage III patients with a less favourable prognostic profile.

Thus the present invention pertains to the determination of the biomarkers (e.g. TIMP-1 and CEA and optionally at least one additional parameter) following neo-adjuvant and/or adjuvant therapy, thereby obtaining information on the efficacy of the given neo-adjuvant or adjuvant treatment. In the case of information that indicates lack of efficacy of the given neo-adjuvant and/or adjuvant treatment, the patients should be offered a second course of neo-adjuvant and/or adjuvant treatment with anti-cancer treatment modalities not exhibiting cross resistance patterns with the anti-cancer treatment modalities that were administered as first line treatment. Such information on the efficacy of neo-adjuvant or adjuvant treatment can in accordance with the present invention be obtained by measuring the biomarker levels at more than one time point (e.g. at a first and a second time point) in the course of the disease, the second time points may be days or months after the initiation of treatment. When comparing biomarker levels between time points, a risk assessment for lack of therapy efficacy of the given therapy can be estimated

By the method of the invention high risk stage II CRC patients who today do not receive adjuvant treatment can now be identified and offered adjuvant treatment. At the same time, the method can identify low risk stage III CRC patients who could be offered less toxic adjuvant therapy or no adjuvant therapy. The invention thus in one aspect relates to a method for improving the survival of stage II CRC patients, the method comprising CEA and TIMP-1 measurements and optionally at least one additional parameter in a preoperatively collected blood sample and then entering these values into a predefined algorithm which provides a risk assessment for the individual patient regarding disease recurrence. Treatment could be cytotoxic chemotherapy and/or biological treatment, e.g. EGFr antibodies, VEGF antibodies, kinase inhibitors or immunotherapy, with the aim of increasing the chance of the patients to survive the cancer disease.

A second aspect of the invention relates to a method for decreasing toxicity of adjuvant treatment without affecting survival probabilities in stage III CRC patients, the method comprising CEA and TIMP-1 measurements in a preoperatively collected blood sample and then entering these values into a predefined algorithm which provides a risk assessment for the individual patient regarding disease recurrence. Patients with a low likelihood of disease recurrence or disease related death could be offered less toxic or no adjuvant treatment while patients with high likelihood of disease recurrence, should be offered adjuvant systemic therapy with the most effective drugs and drug combinations available.

In another embodiment the present invention pertains to a method where the effect of neo-adjuvant or adjuvant treatment can be monitored in the individual patient, the method comprising CEA and TIMP-1 measurements in a blood sample obtained after termination of adjuvant therapy of stage II and stage III CRC patients. The CEA and TIMP-1 values are entered into a predefined algorithm which provides a risk assessment for the individual patient regarding disease recurrence or disease related death after adjuvant treatment. Patients with a low likelihood of disease recurrence or disease related death after adjuvant treatment could be offered surveillance while patients with a high likelihood of disease recurrence or disease related death after adjuvant treatment should be offered additional adjuvant systemic therapy with the most effective drugs (e.g. more aggressive drugs) and drug combinations available.

Kits

The present invention further provides kits for application of the methods according to the invention.

Accordingly, an aspect of the present invention relates to a kit for determining whether a gastrointestinal cancer patient is likely to experience a disease recurrence, said kit comprising
a) reagents for determining the total concentration of TIMP-1 in a pre-operative plasma sample of said patient, and
b) reagents for determining the concentration of CEA in a pre-operative body fluid sample of said patient, and optionally
c) reagents for determining the concentration of one or more additional tumour markers in a pre-operative body fluid sample of said patient.
d) reagents for determining the concentration of TIMP-1 and CEA and one or more additional tumour markers in a post-treatment body fluid sample of said patient.

In one embodiment the kit comprises at least antibody against TIMP-1, CEA or said additional tumour marker.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting figures and examples.

### FIGURE LEGENDS

FIG. 1

Colorectal cancer (CRC) stage II and III populations.

This figure illustrates that among the population of patients with stage II CRC, none of the patients will routinely receive adjuvant systemic therapy although it is known that approximately 15% of these patients will experience a disease recurrence over 5 years postoperatively. Of a population of patients with stage III CRC, approximately 30% are cured by the initial surgical procedures although all patients with stage III CRC are treated with adjuvant systemic anti-cancer therapy. The figure illustrates that the present invention allows for the identification of the prognostic group of stage II and III who should and who should not be offered adjuvant systemic anti-cancer therapy.

FIG. 2.

The hazard ratios based on the multivariable model as a function of TIMP-1.

The hazard ratios based on the multivariable model are shown as a function of TIMP-1 for CEA levels equal to 5, 20 and 50 ng/ml. The baseline TIMP-1 level was set to 80 ng/ml and CEA to 2 ng/ml.

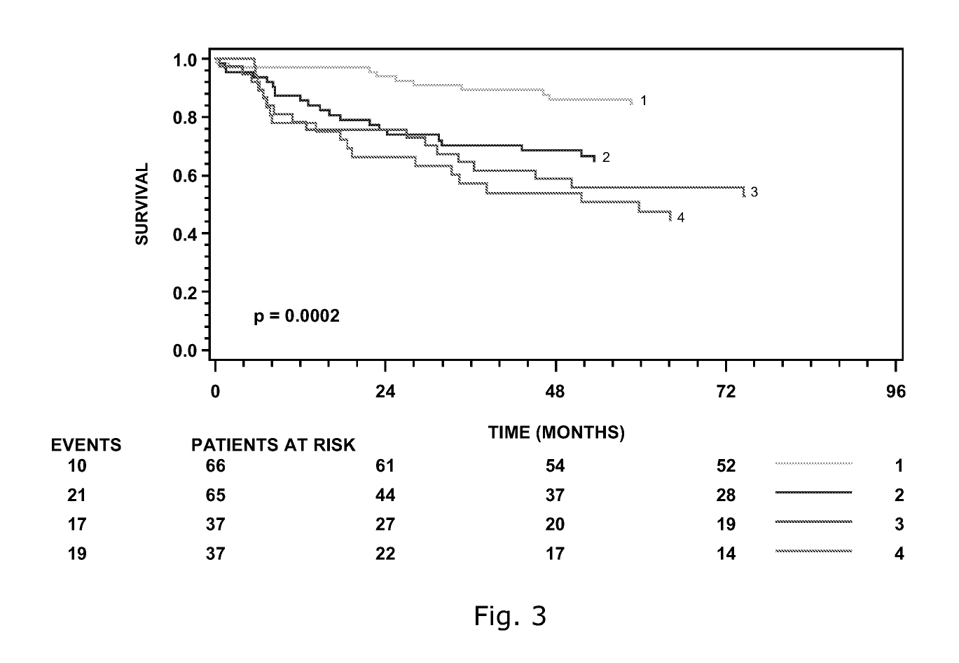

FIG. 3.

Kaplan-Meier estimates of disease free survival for Dukes' B patients. The Kaplan-Meier estimates are shown for CC (1 and 2) and RC (3 and 4) patients. The respective median levels of the index based on the multivariable results have been used to dichotomize the CC and RC patients. “1” denotes CC patients with index level below the median and “2” those above. Similarly “3” denotes levels below the median for RC patients and “4” those above. The number of events for each stratum is shown to the left below the axis and the number at risk at entry, 24 months, 48 months and 72 months are also shown below the axis.

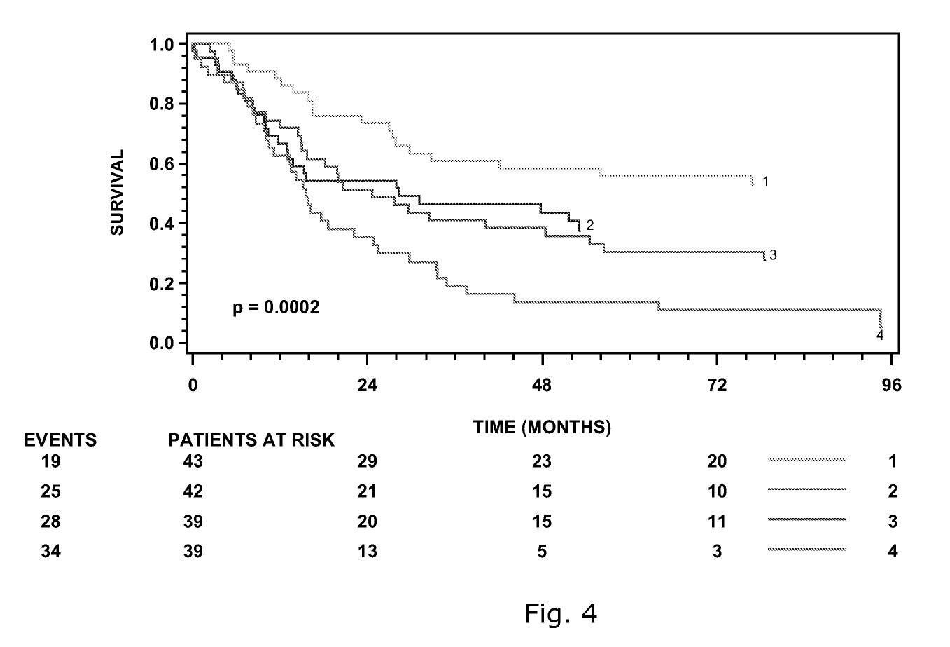

FIG. 4.

Kaplan-Meier estimates of disease free survival for Dukes' C patients. The Kaplan-Meier estimates are shown for CC (1 and 2) and RC (3 and 4) patients. The respective median levels of the index based on the multivariable results have been used to dichotomize the CC and RC patients. “1” denotes CC patients with index level below the median and “2” those above. Similarly “3” denotes an index below the median for RC patients and “4” those above. The number of events for each stratum is shown to the left below the axis and the number at risk at entry, 24 months, 48 months and 72 months are also shown below the axis.

FIGS. 5 and 6

Disclose general, preferred steps in a process being carried out in preferred embodiments of the invention in order to reach an indication on whether it is likely that a given patient (in FIG. 5 the patient is at stage II and in FIG. 6, the patient is at stage III) will experience disease recurrence if the treatment scheme the patient in question follows is maintained.

Tables

TABLE 1
Median and range levels of plasma TIMP-1 and serum CEA
in relation to gender, tumor location, and Dukes' stage.

| | TIMP-1 ng/ml | CEA ng/ml |
|---|---|---|
| Females (n = 166) | 134 (66-550) | 2.8 (0.4-428) |
| Males (n = 256) | 131 (54-510) | 3.2 (0.3-1103) |
| Colon cancer (n = 235) | 147 (58-550) | 3.1 (0.5-1103) |
| Rectal cancer (n= 187) | 119 (54-510) | 2.9 (0.3-121) |
| Dukes' stage A (n = 54) | 116 (63-420) | 1.7 (0.5-1103) |
| Dukes' stage B (n = 205) | 136 (58-550) | 2.9 (0.3-428) |
| Dukes' stage C (n = 163) | 132 (58-411) | 3.5 (0.4-379) |

Number of patients is shown for each patient category.

TABLE 2
Multiple Cox analysis of disease free survival

| | HR | 95% CI | p value |
|---|---|---|---|
| Log2 CEA | 1.1 | 1.0-1.2 | 0.0053 |
| Log2 TIMP-1 | 1.5 | 1.1-2.0 | 0.0071 |
| CEA/TIMP-1 profile | | | |
| Dukes' B vs. A | 4.2 | 2.5-8.0 | 0.0001 |
| Dukes' C vs. B | 2.5 | 1.9-3.5 | 0.0001 |
| Patient age | 1.1 | 0.9-1.3 | 0.24 |
| Gender | 1.2 | 0.9-1.6 | 0.29 |
| Rectum vs. colon | 2.4 | 1.8-3.3 | 0.0001 |

### EXAMPLES

### Example 1

This example describes a clinical study that was performed to test the prognostic value of the combination of plasma TIMP-1 and serum CEA in patients with colorectal cancer.

Background

Present estimations indicate that approximately 600,000 individuals will be diagnosed with colorectal cancer (CRC) and 260,000 will dye from CRC pro annum in Europe and USA. At the primary diagnosis 80% of the patients will undergo intended curative resection, while the remaining 20% will only be offered palliative treatment. However, among the curatively resected patients about 40-45% will develop recurrent disease within the next five years, resulting in an overall long-term survival of only 50%. Stage dependent 5-year overall survival in curatively resected patients is in stage I: 90%-95%, stage II: 70%-75%, stage III: 35%-40%, which indicates that despite lack of clinical, histological and biochemical evidence the disease has disseminated before or during surgery in a significant number of patients. The efficacy of adjuvant therapy to patients with stage III disease is well established, but due to the limited benefit in stage I and II disease these patients are not routinely offered adjuvant treatment (4-6). As mentioned above, still some 25%-30% of patients with stage II disease are, however, at risk of developing recurrence and possibly such patients might have benefit of some form of adjuvant treatment. The challenge is how to identify these “at risk” patients and offer them adjuvant treatment and at the same time to avoid offering unnecessary adjuvant treatment to the 70%-75% of the patients, who are already cured by primary surgery. Present risk factors for selection of patients with stage II CRC to chemotherapy are: low numbers of lymph nodes in the resected specimen, T4 stage (TNM classification), large bowel perforation, and poor histological differentiation grade.

As stated above, a fair amount of the patients with stage III disease seem to have no benefit of adjuvant treatment, and the challenge appears to be the opposite of that in stage II disease—to identify those stage III patients, who have a low risk of recurrence and thus, who should not being offered adjuvant treatment.

Serum CEA has been recommended by individual researchers, ASCO and EGTM (European Group on Tumour Markers) as a prognostic biomarker in curatively resected CRC patients. Still the recommendation is limited to identify patients, who may have benefit of re-operation or palliative treatment modalities or CEA may be used to monitor patients postoperatively with the aim of detecting liver metastasis at a point in time where they can be surgically resected. As CEA is the only recommended soluble marker in CRC, it would be preferable if new serological biomarkers that could be additive to CEA could b identified. One such marker could be Tissue Inhibitor of MetalloProteinases-1 (TIMP-1), which inhibits the activity of active Matrix Metalloproteinases (MMP), in particular MMP-2 and MMP-9. Based on this inhibition it has been presumed that high tumor levels of TIMP-1 protein would be associated with favourable patient outcome. However, a large number of studies have shown that high levels of TIMP-1 in plasma associates with short time to cancer disease recurrence and short cancer patient survival. The mechanisms linking poor prognosis and high TIMP-1 levels may be related to the fact that TIMP-1 has other actions than inhibition of MMP's, e.g. stimulation of cell growth, regulation of angiogenesis, and inhibition of apoptosis.

The purpose of the present study was to determine the value of the combination of serum CEA and plasma TIMP-1 levels as a prognostic index in patients having undergone intended curative resection of primary CRC. Such a prognostic index might be useful to stratifying patients into different risk groups thereby allowing for differentiated adjuvant treatment.

The present inventors followed the REMARK guidelines for reporting the study when ever applicable.

Patients and Methods

Patients

The patient cohort (N=422) was scheduled for elective large bowel operation based on biopsy verified or barium enema-suspected primary colorectal adenocarcinoma. All patients were included from April 1991 through August 1993 and were randomised to receive the histamine-2 receptor antagonist Ranitidine or placebo in a double blind, clinically controlled, multicenter (20 Danish surgical departments) study. The study was conducted in accordance with the Helsinki II declaration and was approved by the Central National Ethics Committee. The approval also included collection of plasma and serum samples for subsequent analysis of biological markers. All patients were without clinical signs or symptoms of infectious disease, and none had been treated with systemic steroids, antibiotics, or antiviral drugs within 2 weeks prior to resection. Patients with severe concurrent illness, such as HIV infection or prior cancer, were not included in the study. All patients had histologically verified adenocarcinoma of the colon or rectum. The diagnosis and stage of CRC was established pathologically from the resected primary tissue and from biopsies of involved lymph nodes or distant metastases when present. The stage of disease was classified according to Dukes' staging.

The study was performed at a time where radio- and/or chemotherapy were not part of the standard offer in Denmark to patients with CRC. Therefore, none of the patients received such treatment modalities either before or after surgery. The patients were randomised to receive Ranitidine or placebo twice daily for up to five years. In the original study there was no effect of Ranitidine on overall long-time survival. The survival benefit was observed only in a selected subgroup of patients. All patients were followed in the outpatient clinic and at the first visit one month after the resection all perioperative data, including operation procedure, stage and location of the disease, and postoperative complications were recorded in the case report form (CRF). At subsequent visits every third month up to five years the patients underwent routine blood analysis and clinical examination to identify recurrence or metastasis in otherwise curatively resected patients. Endoscopy, US and/or barium enema were part of the examination procedures when appropriate.

Blood Sampling

Before surgery and before the first infusion of Ranitidine/placebo all patients had blood collected with minimal stasis to prevent platelet activation from an antecubital vein. The blood was processed according to a SOP and serum and plasma were kept frozen at −80° C. until analysis. Corresponding serum and plasma samples were available from 589 patients.

TIMP-1 and CEA

When all the blood samples were collected CEA protein levels were determined in serum using a solid-phase, chemiluminescent EIA kit (Immulite CEA; Diagnostic Products Corporation, Los Angeles, Calif.). The assay has a detection limit of 0.2 ng/ml, recovery of approximately 100%, and intra- and interassay CV of 5%, and 6% respectively. TIMP-1 protein levels were determined in EDTA plasma using an in-house, rigorously validated kinetic rate ELISA demonstrating low intra- and interassay CV. Plasma TIMP-1 determination did undergo a thorough pre-, per- and postanalytical validation. Due to cellular disintegration and release of TIMP-1 from platelet and granulocyte granules during coagulation it is recommended that TIMP-1 is determined in EDTA plasma. The ELISA determines total TIMP-1 (the free form plus the form in complex with MMP). In brief, affinity-purified sheep polyclonal anti-TIMP-1 antiserum was used as a capture antibody in a 96-well microtiter plate. A murine monoclonal anti-TIMP-1 IgG1 (MAC15) was used for detection, and a rabbit antimouse immunoglobulin/alkaline phosphatase conjugate (Dako, Glostrup, Denmark) was the secondary antibody that enabled the kinetic rate assay. Rate measurements were collected automatically over a 1-h period in a Ceres 900 plate reader (Bio-Tek Instruments, Winooski, Vt.). A four-parameter fitted standard curve was generated using Kineticalc II software (Bio-Tek), from which the total TIMP-1 concentration of each sample was calculated. In the present study the intra-assay CV was 5.3% and the interassay CV was 7.4%. All samples were determined in duplicate and the mean value used in the subsequent calculations.

Statistics

Rank statistics were used to calculate correlation coefficients and to test hypotheses on location. Tests of independence were done using the chi-square test. The levels of TIMP-1 and CEA were log-transformed (log 2) and treated as continuous variables for the uni- and multivariate analyses of disease free survival. The clinical covariates (Dukes' stage stage, localization, age and gender) were scored using indicator variables. The Kaplan-Meier method was used to estimate survival probabilities, and the log-rank test was used to test for equality of strata. The Cox proportional hazards model was applied for univariate analysis as well as for DFS in a multivariate analysis. DFS is defined from the time of surgery to the time of diagnosis of local recurrence or distant metastasis or death by cancer. Tests for interaction (covariance) between the biomarkers and clinical covariates were also included in this analysis. The assumption of proportional hazards and linearity of the continuous covariates were assessed using the residuals. A prognostic index was constructed based on the regression coefficients from the multivariate analysis and adjusted for the baseline covariates. The significance level was set to 5%. The SAS® software package (version 9.1; SAS Institute, Cary, N.C.) was used to manage patient data and to perform all statistical analyses.

Results

The clinical study enrolled 589 patients with serum and plasma samples available among whom 167 had stage 1V CRC. The final number of CRC patients with stage I-III disease was thus 422. The diagnoses were Dukes' stage A: 54 patients; Dukes' stage B: 205 patients; Dukes' stage C: 163 patients, which comprises the present study cohort. Colon cancer was diagnosed in 235 and rectal cancer in 187 of the patients, and 166 were females and 256 males. The median age was 69 (range 33-91) years. The follow-up period was 7.9 (range 5.5-9.1) years. An event was recorded in 186 patients; 75 had local recurrence, 75 had distant metastases and 28 had both local recurrence and distant metastases. Thirty-six patients died from their cancer without a registered recurrence.

The median serum CEA level was 3.0 (range 0.3-1103.0) ng/ml; 283 patients (67.1%) had CEA levels 5 ng/ml, 90 patients (21.3%) had CEA levels >5 ng/ml and 20 ng/ml and 49 patients (11.6%) had CEA levels >20 ng/ml. The median plasma TIMP-1 level was 131.5 (range 53.7-549.8, first quartile 105.6 ng/ml, third quartile 173.5 ng/ml) ng/ml. Plasma TIMP-1 levels increased with age: rₛ=0.436; p<0.0001, while age had no relation to serum CEA levels. The correlation between serum CEA and plasma TIMP-1 was: rₛ=0.20; p<0.0001. The levels of the markers in relation to gender, stage and location of disease are shown in Table 1.

A multivariable Cox analysis of DFS including serum CEA, plasma TIMP-1, stage of disease, age, gender, and location of disease showed that both serum CEA and plasma TIMP-1 as continuous variables were independent predictors for DFS: serum CEA HR: 1.1; 95% CI: 1.0-1.2; p=0.0053 and plasma TIMP-1 HR: 1.5; 95% CI: 1.1-2.0; p=0.0071. High levels of both markers were related to short DFS. In addition stage of disease and location in rectum were independently related to risk of recurrence, while age and gender were not. The results of the multivariable analysis are shown in Table 2. Treatment with Ranitidine was not significant in the multivariable model (p=0.60) nor could significant interactions with CEA and TIMP-1 be demonstrated (data not shown).

An index for the relation between serum CEA and plasma TIMP-1 adjusted for the clinical baseline variables was established and defined as: 0.11×Log 2 (CEA)+0.41×Log 2 (TIMP-1). FIG. 2 shows the HR for recurrence with variable TIMP-1 at the X-axis and with CEA set to 5, 20, and 50 ng/ml, respectively. The baseline values for the calculations of hazard ratios were set to 80 ng/ml for TIMP-1 and 2 ng/ml for CEA. As an example this index shows a 51% increase in the HR for a given CEA level if the TIMP-1 level is doubled. Based on this index, the estimated DFS probabilities for Dukes' B patients stratified by CC and RC are shown in FIG. 3. The respective median level of the index has been used to dichotomize the patients for CC and RC. As can be seen form FIG. 3 an event (DFS) for CC patients with marker index below the median is only registered in a very few patients 24 months after the resection while this number is much higher in patients with marker index over the median. The difference between RC patients with low versus high index is not as pronounced. A similar Figure for Dukes' C patients is shown in FIG. 4.

Discussion

The results of the present study show that both preoperative serum CEA levels and plasma TIMP-1 levels yielded significant, independent information on DFS of patients, who underwent curative resection for primary CRC. For both proteins high levels were related to poor patient outcome. The independence of these two markers shown in the multivariable analysis implied that, when including both biomarkers in an index, an increased identification of patients at risk of developing stage- and location-independent recurrent disease or distant metastasis could be obtained. Specifically, this was pronounced for stage II patients with CC, while the index had limited value among stage II patients with RC (FIG. 3). However, the index was also able to differentiate between stage III CC patients with low or high risk of recurrence and stage III RC patients with low or high risk of recurrence (FIG. 4).

This study included a patient cohort that had not received neoadjuvant or adjuvant chemo- and/or radiotherapy. The patients were randomised to receive Ranitidine or placebo twice daily for up to five years. In the original study there was no effect of Ranitidine on overall long-time survival. A small survival benefit was observed only in a selected subgroup of patients, but there was no effect of Ranitidine on the results from the present study.

The present recommendations for use of CEA in primary CRC are limited to identify patients, who should be followed by repeated CEA measurements allowing for early surgical or medical intervention in the case of recurrent disease. Previous results from the complete cohort of patients did not show that preoperative CEA carried any prognostic information. This may be explained by the fact that Dukes' stage D patients were included in these calculations. Restrictions of CEA measurements to Dukes' stage A, B and C patients in the present study showed that CEA had stage-independent prognostic information including all three stages.

Previous reports have shown that preoperative plasma TIMP-1 levels carried stage-independent prognostic information in CRC patients; high levels of TIMP-1 are related to poor prognosis in a stage independent fashion. These results have subsequently been validated in a number of studies. Due to lack of any evidence of a common biological significance of CEA and TIMP-1, the present inventors raised the hypothesis that the addition of preoperative plasma TIMP-1 to preoperative serum CEA would improve the identification of patients at risk of recurrence. Thus, CEA and TIMP-1 would have to yield independent prognostic information and this information would have to be stage independent as well.

The present study supports our hypothesis, since it was shown that the combination of these two proteins did improve the prognostic stratification that could be obtained with each of the proteins. Also, this information was stage independent. The advantage of the present patient cohort has been that none of the patients received adjuvant chemo- and/or radiotherapy. Thereby the DFS recordings are not biased by concomitant adjuvant treatment.

The location of disease in the rectum carries a very poor and independent prognosis in the present study. Patients with RC were operated at a time before introduction of the TME (Total Mesorectal Excision, 31) approach of the rectum and they did not receive any pre- and/or postoperative radiotherapy. More recent results suggest that CC and RC patients may have similar DFS, while CC patients still have a better OS than RC).

In conclusion, a prognostic index based on preoperative serum CEA and plasma TIMP-1 will be useful in future identification of patients with stage II disease, who should be offered adjuvant treatment. Furthermore, this index will be used to introduce differentiate treatment e.g. less or more aggressive systemic therapy among stage III CRC patients. However, the present results must be validated in independent patient cohorts where proper adjuvant therapy has been offered to the relevant patients—see Example 2.

### Example 2

This example describes a prospective validation of the combination of plasma TIMP-1 and serum CEA as prognostic markers in Dukes stage B colorectal cancer patients and in Dukes stage C colorectal cancer patients, respectively.

Background

Example 1 shows that simultaneous measurements of plasma TIMP-1 and serum CEA yields highly statistically significant prognostic information in both Dukes stage B patients and in Dukes stage C patients when analysing blood samples obtained preoperatively from patients scheduled for colorectal cancer surgery, (Example 1). The prognostic stratification obtained by the combined measurements of TIMP-1 and CEA is stronger than what can be obtained by only measuring one of these proteins. The inventors have now undertaken a validation study to test the hypothesis, that combined measurements of TIMP-1 and CEA in a preoperatively obtained blood sample will provide better prognostic stratification of colorectal cancer patients than measuring only one of these proteins.

Patients and Methods

A total of 337 patients undergoing surgery for colon or rectal cancer were included prospectively and consecutively in the study. Inclusion criteria's were: histologically verified adenocarcinoma of the rectum or colon; preoperatively collected blood sample available; signed informed consent to use blood samples for tumor marker studies.

Based on availability of plasma and serum samples, the final number of patients included in the present study was 321 with 215 colon cancers and 106 rectal cancers with 26 recurrences in the colon cancers and 29 recurrences among the rectal cancers. There were 101 stage II colon cancers and 32 stage II rectal cancers with 8 and 7 recurrences, respectively. In the group of stage III patients there were 57 colon cancers and 35 rectal cancers with 17 and 21 recurrences, respectively. The median age of the patients at time of surgery was 72 years (range 35-94 years). The observation time was 5.5 years (range 3.9-7.4 years).

For each patient additional information on tumor grade, lymphatic or vascular vessel invasion and adjuvant treatment was available.

Blood Sampling

All blood samples were collected preoperatively and in the case of rectal cancer patients also before any irradiation treatment. The blood samples were immediately processed for EDTA plasma and for serum. The samples were stored at −80° C. until analysed for TIMP-1 and CEA content.

TIMP-1 and CEA ELISA

All assays were performed without knowledge of the clinicopathological data. A thoroughly validated ELISA was used to measure the TIMP-1 levels in plasma (Holten-Andersen et al., Br J Cancer 1999). In brief, an affinity purified sheep polyclonal anti-TIMP-1 antibody was used to capture TIMP-1 in the specimens. MAC15, a murine monoclonal anti-TIMP-1 IgG1 was used to detect the TIMP-1, and a rabbit anti-mouse immunoglobin/alkaline phosphatase conjugate (No. D0314; DAKO, Denmark) was used for the kinetic rate measurement. The rate measurements were collected every 10 min over a 1 hour period. KinetiCalc II software (Bio-Tek Instruments, Winooski, Vt.) was used to determine the TIMP-1 levels in the specimens. The MAC15 antibody recognises both free and MMP-bound TIMP-1 and the assay can therefore be considered as a total TIMP-1 assay.

Before performing the assay, all specimens were thawed at 37° C. The intra- and inter assay variations were below 10%.

The CEA analyses were performed according to the instructions provided by the vendor. The CEA assay was obtained from IBL, Hamburg, Germany

Results

All analysed patient samples contained TIMP-1 levels above the detection limit of the assay. The median plasma TIMP-1 values for all curably resected patients (stage I-III) was: 153.52 mg/ml (range: 57.43-588.10 mg/ml); When analysing the TIMP-1 values in the stage II and in the stage III patients separately, it was found that stage II colon cancer patients had a plasma TIMP-1 median level of 155.31 ng/ml (range 68.35-530 ng/ml). The corresponding numbers in stage II rectal cancer patients were (median: 158.88; range 76.28-344.63). In the stage III colon cancer patients the plasma TIMP-1 median level was 157.54 (range: 72.49-370 and 149.22 (range 57.43-588.10) in the stage III rectal cancer patients. Thus, the plasma TIMP-1 levels in stage II patients were not significantly different from the plasma levels of TIMP-1 in stage III patients or levels found in the whole patient cohort (p>0.05). Similarly, no significant differences in plasma TIMP-1 levels were observed between colon and rectal cancer patients.

Plasma TIMP-1 levels associated significantly with overall survival of the whole patient population when treating TIMP-1 (logₑTIMP-1) as a continuous variable. Including the stage II and stage III patient cohort only (Cox proportional hazards model, P<0.0001) the estimated hazard ratio was 3.12 (95% CI, 1.87-5.19)) indicating the increase in hazard for patients differing by one unit on the logₑ scale. The corresponding figures for the association between logₑ TIMP-1 levels and disease free survival were: Cox proportional hazards model, P<0.0009 with an estimated hazard ratio of 2.17 (95% CI, 1.37-3.44) indicating the increase in hazard for patients differing by one unit on the logₑ scale. For both clinical end-points, patients with higher plasma TIMP-1 values had shorter survival and shorter disease free survival. The Kaplan-Meier curves for overall survival for the stage II and stage III patient cohort when the patients were stratified into plasma TIMP-1 tertiles were constructed. A test for trends showed a significant difference (p<0.0004) in overall survival between the three strata. Thus, this analysis confirmed that increasing plasma TIMP-levels are associated with a continuously increasing risk of dying. The survival probability at 24 months was above 80% in patients with plasma TIMP-1 levels below the first stratum, while being approximately 60% for patients with plasma TIMP-1 levels above the third stratum. Then Kaplan-Meier curves for disease free survival for the whole patient cohort were constructed. Again, the patients were stratified according to plasma TIMP-1 tertiles. A test for trends revealed a statistically significant difference (p<0.004) in disease free survival in the three patient strata with almost double as many events in the high plasma TIMP-1 patients as compared with patients with plasma TIMP-1 levels below the lowest tertile.

Multivariate Cox regression analyses of overall survival and disease free survival data were performed including age, gender, tumor localization, stage of disease, differentiation grade, lymphatic or vascular vessel invasion, adjuvant treatment and logₑ TIMP-1 values. 312 patients with 116 deaths were eligible for overall survival analyses and 268 patients with 100 recurrences were eligible for disease free survival analyses. Age, stage of disease and loge TIMP-1 values were the only variables that were retained in the multivariate model for overall survival as well as for disease free survival. High plasma TIMP-1 levels were independently associated with shorter overall survival (HR 2.35 (95% CI. 1.51-3.66; p=0.0006) and disease free survival HR 1.86 (95% CI: 1.12-3.8; p=0.017).

The CEA values were added into the model and it was found that CEA was an independent predictor of overall survival and disease free survival being independent of stage of disease and TIMP-1. Thus, for each stage of disease, the prognostic impact of plasma TIMP-1 values and serum CEA values could be added resulting in a prognostic stratification of stage II patients and of stage III patients being superior to the stratification that could be obtained by only measuring serum CEA or by only measuring plasma TIMP-1

Discussion

This prospective validation study confirmed previous results demonstrating a highly significant association between plasma TIMP-1 levels and serum CEA levels and disease free survival and overall survival of stage II and stage III colorectal cancer patients. This study thus brings the combination of plasma TIMP-1 and serum CEA measurements to the next level of evidence for its clinical use as a prognostic marker in curably resected CRC patients.

### Example 3

CRC is a disease which is primarily seen in the elderly population. The clinical significance of a high risk of disease recurrence in stage II CRC patients might therefore also depend on the age, co-morbidity and performance status of the individual patient. For example, a 60 year old patient with stage II disease, good performance status and a HR of >2 for recurrence will most probably be offered adjuvant treatment while a similar HR in a 72 year old patient with severe co-morbidity and low performance status might not result in adjuvant treatment. Therefore, it might be more clinically relevant to calculate a risk score for the individual CRC patient and then take any other parameter into account when discussing additional treatment with the patient. Such a risk score can be generated by including plasma TIMP-1 and serum CEA measurements in preoperatively collected plasma sample.

### REFERENCES

Bogaerts J, Cardoso F, Buyse M, Braga S, Loi S, Harrison J A, Bines J, Mook S, Decker N, Ravdin P, Therasse P, Rutgers E, van 't Veer L J, Piccart M; TRANSBIG consortium. Mol Oncol 2007, 1:246-251.
Holten-Andersen, M. N., Murphy, G., Nielsen, H. J., Pedersen, A. N., Christensen, I. J., Høyer-Hansen, G., Brünner, N. & Stephens, R. W.: Quantitation of TIMP-1 in plasma of healthy blood donors and patients with advanced cancer. Br. J. Cancer 1999, 80:495-503.
Holten-Andersen, M., Stephens, R. W., Nielsen, H. J., Murphy, G., Christensen, I. J., Stetler-Stevenson, W. & Brünner, N.: High preoperative plasma TIMP-1 levels are associated with short survival of patients with colorectal cancer. Clin Cancer Res., 2000, 6: 4292-4299.
Holten-Andersen, M N., Christensen, I J., Nielsen, H J., Stephens, R W., Jensen, V., Nielsen, O H., Sørensen, S., Overgaard, J., Lilja, H., Harris, A., Murphy, G., and Brünner N. Total levels of tissue inhibitor of metalloproteinases 1 in plasma yield high diagnostic sensitivity and specificity in patients with colon cancer. Clin. Cancer Res. 2002, 8: 154-164.
Holten-Andersen, M N., Christensen, I J., Nielsen, H J., Lilja, H., Murphy, G., Jensen, V., Brünner, N., Piironen, T. Measurement of the non-complexed free fraction of tissue inhibitor of metallopoteinases 1 in plasma by immunoassay. Clin. Chem. 2002, 48:1305-1313
Holten-Andersen M N., Christensen I J., Nilbert N., Bendahl P O., Nielsen H J., Brünner N and Fernebro E. Association between preoperative plasma levels of tissue inhibitor of metalloproteinases 1 and rectal cancer patient survival: A validation study. Eur. J. Cancer. 2004, 40: 64-72.
Ishida-H, Murata-N, Hayashi-Y, Tada-M and Hashimoto-D. Surg. Today 2003, 33: 885-892.

1. A method for determining whether a gastrointestinal cancer patient is likely to experience a disease recurrence or disease related death, said method comprising
a) determining the total concentration of TIMP-1 in a pre-operative plasma sample of said patient,
b) determining the concentration of CEA in a pre-operative body fluid sample of said patient,
c) combining the concentration of TIMP-1 with the concentration of CEA to result in a combined TIMP-1/CEA parameter,
d) indicating the patient as likely to experience a disease recurrence or disease related death if the combined TIMP-1/CEA parameter is at or above a predefined combined TIMP-1/CEA discriminating value, and
e) indicating the individual as unlikely to experience a disease recurrence or disease related death if the combined TIMP-1/CEA parameter is below said predefined TIMP-1/CEA combined discriminating value.

2. A method for determining whether a gastrointestinal cancer patient is likely to experience a disease recurrence or disease related death, said method comprising
a) determining the total concentration of TIMP-1 in a pre-operative plasma sample of said patient,
b) determining the concentration of CEA in a pre-operative body fluid sample of said patient,
c) determining at least one additional parameter pre-operatively of said patient,
d) combining the concentration of TIMP-1 with the concentration of CEA and the at least one additional parameter to result in a combined additional parameter,
e) indicating the patient as likely to experience a disease recurrence or disease related death if the combined additional parameter is at or above a predefined combined additional discriminating value, and
f) indicating the individual as unlikely to experience a disease recurrence or disease related death if the combined additional parameter is below said predefined combined additional discriminating value.

3. A method for determining whether a gastrointestinal cancer patient is likely to experience a disease recurrence or disease related death following neo-adjuvant or adjuvant anti-cancer therapy, said method comprising
a) determining at a first time point the total concentration of TIMP-1 in a pre-operative plasma sample of said patient,
b) determining at said first time point the total concentration of CEA in a pre-operative plasma sample of said patient,
b) combining the concentration of TIMP-1 with the concentration of CEA to result in a combined TIMP-1/CEA parameter,
c) indicating the patient as likely to experience a disease recurrence or disease related death if the combined TIMP-1/CEA parameter is at or above a predefined combined TIMP-1/CEA discriminating value, and
d) indicating the individual as unlikely to experience a disease recurrence or disease related death if the combined TIMP-1/CEA parameter is below said predefined TIMP-1/CEA combined discriminating value,
e) if the individual is likely to experience a disease recurrence or disease related death determining at a second time point the total concentration of TIMP-1 in a post-operative plasma sample of said patient,
f) determining at said second time point the total concentration of CEA in a post-operative plasma sample of said patient,
g) combining the concentration of TIMP-1 with the concentration of CEA to result in a combined TIMP-1/CEA parameter,
h) indicating the patient as likely to experience a disease recurrence or disease related death if the combined TIMP-1/CEA parameter is at or above a predefined combined TIMP-1/CEA discriminating value, and
i) indicating the individual as unlikely to experience a disease recurrence or disease related death if the combined TIMP-1/CEA parameter is below said predefined TIMP-1/CEA combined discriminating value.

4. A method for determining whether a gastrointestinal cancer patient is likely to experience a disease recurrence or disease related death following neo-adjuvant or adjuvant anti-cancer therapy, said method comprising
a) determining at a first time point the total concentration of TIMP-1 in a pre-operative plasma sample of said patient,
b) determining at said first time point the concentration of CEA in a preoperative body fluid sample of said patient,
c) determining at said first time point at least one additional parameter pre-operatively of said patient,
d) combining the concentration of TIMP-1 with the concentration of CEA and the at least one additional parameter to result in a combined additional parameter,
e) indicating the patient as likely to experience a disease recurrence if the combined additional parameter is at or above a predefined combined additional discriminating value,
f) indicating the individual as unlikely to experience a disease recurrence or disease related death if the combined additional parameter is below said predefined combined additional discriminating value,
g) if the individual is likely to experience a disease recurrence or disease related death determining at a second time point the total concentration of
TIMP-1 in a post-operative plasma sample of said patient,
h) determining at said second time point the concentration of CEA in a post-operative body fluid sample of said patient,
i) determining at said second time point at least one additional parameter post-operatively of said patient,
j) combining the concentration of TIMP-1 with the concentration of CEA and the at least one additional parameter to result in a combined additional parameter,
k) indicating the patient as likely to experience a disease recurrence if the combined additional parameter is at or above a predefined combined additional discriminating value, and
l) indicating the individual as unlikely to experience a disease recurrence or disease related death if the combined additional parameter is below said predefined combined additional discriminating value.

5. A method according to claim 2, wherein the at least one additional parameter is selected from the group consisting of age, gender, general clinical status, tumour localization, stage of disease, number of lymph nodes examined, differentiation grade, lymphatic, vascular vessel invasion, nerve invasion, co-morbidity, adjuvant treatment regime and an additional tumour marker different from any form of TIMP-1 or CEA, in a body fluid sample from the patient.

6. A method according to claim 5, wherein the additional tumour marker is selected from the group consisting of serum or plasma soluble suPAR including its variant forms, serum or plasma CA19.9, serum or plasma CA242, un-complexed TIMP-1, TIMP-1 in complex with specific MMP's, variant forms of CEA, soluble CD63, YKL-40, p66 She, MMP's, ADAM's and kallekreins and combinations thereof.

7. A method according to claim 1, wherein the combining is performed by logistic regression analysis.

8. The method according to claim 1, wherein the concentration of TIMP-1 is selected from the group consisting of the concentration of TIMP-1 in free form, the concentration of TIMP-1 in complex form, and the total concentration of TIMP-1.

9. The method according to claim 8, wherein the total concentration of TIMP-1 is the concentration of free TIMP-1 and TIMP-1 in complex form.

10. The method according to claim 1, wherein said TIMP-1 concentration is selected from the group consisting of the TIMP-1 mRNA concentration of plasma or tumor sample and TIMP-I protein concentration of plasma or tumor sample.

11. The method according to claim 10, wherein the TIMP-1 protein concentration is the TIMP-1 protein concentration of said plasma sample.

12. The method according to claim 1, wherein said CEA concentration is selected from the group consisting of the CEA mRNA concentration of said body fluid sample or tumor tissue and CEA protein concentration of said body fluid sample or tumor tissue.

13. The method according to claim 12, wherein the CEA concentration is selected from the group of the serum CEA concentration of said body fluid sample, the plasma CEA concentration of said body fluid sample, and the CEA concentration in plasma and serum of said body fluid sample or tumor tissue sample.

14. The method according to claim 1, wherein the TIMP-1 concentration and the CEA concentration is determined in the same sample.

15. The method according to claim 1, wherein gastro-intestinal cancer is selected from the group consisting of oesophageal cancers, gastric cancers, small intestine cancers, gall bladder cancers, liver cancers, colon cancers, rectal cancers and pancreatic cancers.

16. The method according to claim 15, wherein the gastro-intestinal cancer is colorectal cancer.

17. The method according to claim 16, wherein said colorectal cancer is selected from the group consisting of stage II colorectal cancer and stage III colorectal cancer.

18. The method according to claim 16 wherein the gastro-intestinal cancer is an adenocarcinoma selected from the group consisting of colon adenocarcinomas and rectal adenocarcinomas.

19. The method according to claim 3, wherein said anti-cancer treatment is selected from the group consisting of neo-adjuvant therapy, adjuvant therapy, and therapy of metastatic disease.

20. The method according to claim 19 wherein said adjuvant treatment is a systemic anti-cancer treatment.

21. The method according to claim 1, wherein the determination of TIMP-1 and/or CEA is performed by means of an immunoassay selected from the group consisting of ELISA, RIA, immunohistochemistry, Western blotting, mass spectroscopy, flow cytometry, antibody mediated pull-down assay and assays for gene aberrations.

22. The method according to claim 1, wherein the concentration of TIMP-1 and/or CEA is determined using a method selected from the group consisting of a histological method, a cytological method, a method of PCR, a method of RT-PCR such as qRT-PCR

23. The method according to claim 1, wherein said body fluid sample is selected from the group consisting of tumor tissue, tumor tissue comprising non-malignant tissue, plasma, serum, urine, faeces, and saliva.

24. A kit for determining whether a gastrointestinal cancer patient is likely to experience a disease recurrence, said kit comprising
a) reagents for determining the total concentration of TIMP-1 in a preoperative plasma sample of said patient, and
b) reagents for determining the concentration of CEA in a pre-operative body fluid sample of said patient, and optionally
c) reagents for determining the preoperative concentration of an additional tumour marker according to the preceding claim 6 in a pre-operative body 20 fluid sample of said patient.

25. The kit according to claim 24, wherein said kit comprises at least antibody against TIMP-1, CEA or said additional tumour marker.

26. A method of identifying recurrence in a postoperative gastrointestinal cancer patient comprising:
receiving a request to identify the predilection for recurrence of gastrointestinal cancer in a postoperative gastrointestinal cancer patient;
receiving a biological sample obtained from said postoperative gastrointestinal cancer patient; contacting a biological sample obtained from said selected patient with a first antibody to TIMP-1 contacting a biological sample obtained from said selected patient with a second antibody to CEA; determining a first value representing the amount of binding of said first antibody;
determining a second value representing the amount of binding of said second antibody; inputting the first and second values into a computer configured to transform said first and second values into a prognostic index using an algorithm expressed by:
0.11×Log 2(CEA)+0.41×Log 2(TIMP-1)
determining whether said solved prognostic index is associated with recurrence of gastrointestinal cancer or remission of gastrointestinal cancer by comparing the solved prognostic index to a database containing a plurality of prognostic indices, wherein some of the indices are associated with a recurrence of gastrointestinal cancer and some of the indices are associated with remission of gastrointestinal cancer; and communicating said determination of whether said solved prognostic index is associated with recurrence of gastrointestinal cancer or remission of gastrointestinal cancer to said person making said request.

27. A computer system comprising processor means, such as a CPU, and storage means, such as RAM, Hard disk drive, the computer system being adapted to perform the method according to any of the preceding claims.
